(19) United States
(12) Patent Application Publication
(10) Pub. No.: US 2026/0364053 A1
(43) Pub. Date: Nov. 19, 2026

(54) HIGH PURITY CD47-SIRP ALPHA PATHWAY INHIBITOR

(71) Applicant: AURIGENE ONCOLOGY LIMITED, Bangalore (IN)

(72) Inventors: Seetharamaiah Setty Sudarshan NAREMADDEPALLI, Bangalore (IN); M H V Ramana RAO, Bangalore (IN); Siva Prasadu GANTA, Bangalore (IN)

(21) Appl. No.: 19/656,534

(22) Filed: Apr. 23, 2026

(30) Foreign Application Priority Data
Apr. 23, 2025 (IN) 202541039211

(51) Int. Cl. A61K 31/4245; C07D 413/12; C07D 413/14

(52) U.S. Cl. CPC A61K 31/4245; C07D 413/12; C07D 413/14

(57) ABSTRACT

The present disclosure relates to a calcium salt of compound of formula (I):

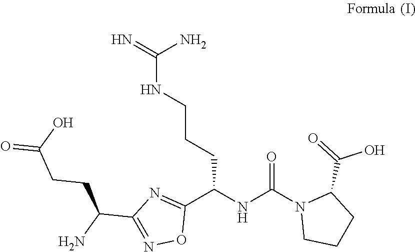

which is substantially free from impurities. The present disclosure further relates to a pharmaceutical composition comprising the said calcium salt of compound of formula (I).

### RELATED APPLICATION

This application claims the benefit of the Indian provisional patent application No. 202541039211, filed on Apr. 23, 2025; the content of which is hereby incorporated by reference in its entirety.

### FIELD OF THE INVENTION

The present disclosure is directed to a calcium salt of compound of formula (I). More particularly, the disclosure relates to a highly pure calcium salt of compound of formula (I):

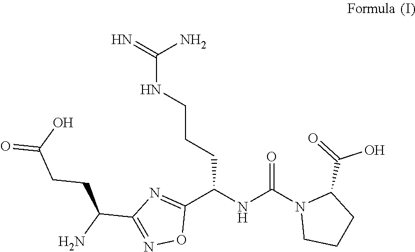

which is substantially free from impurities. The disclosure also relates to a calcium salt of compound of formula (I) which is substantially free from at least one impurity selected from A-1, A-2 and A-3. The present disclosure further relates to a pharmaceutical composition comprising the said calcium salt of compound of formula (I).

### BACKGROUND OF THE INVENTION

CD47 also known as integrin associated protein (IAP), ovarian cancer antigen (OA3), Rh-related antigen and MER6, is a transmembrane protein that, in humans, is encoded by the CD47 gene. CD47 belongs to the immunoglobulin superfamily and partners with membrane integrins and also binds the ligands thrombospondin-1 (TSP-1) and signal-regulatory protein alpha (SIRPα). CD47 is best known for its pivotal role in preventing phagocytic removal of healthy cells by binding to phagocyte-expressed signal regulatory protein alpha (SIRPα). SIRPα, an inhibitory protein expressed on macrophages, once triggered, suppresses phagocytosis of CD47-expressing cells. This CD47/SIRPα axis is an important homeostatic mechanism preventing removal of healthy normal cells that express CD47. Reversely, down-regulation of CD47 on damaged, aged and superfluous cells ensures their timely removal.

CD47 is expressed on virtually all non-malignant cells, and blocking the CD47 or the loss of CD47 expression or changes in membrane distribution can serve as markers of aged or damaged cells, particularly on red blood cells (RBC). Alternatively, blocking SIRPα also allows engulfment of targets that are not normally phagocytosed, for those cells where pre-phagocytic signals are also present. CD47 is a broadly expressed transmembrane glycoprotein with a single Ig-like domain and five membrane spanning regions, which functions as a cellular ligand for SIRPα with binding mediated through the NH₂-terminal V-like domain of SIRPα. SIRPα is expressed primarily on myeloid cells, including macrophages, granulocytes, myeloid dendritic cells (DCs), mast cells, and their precursors, including hematopoietic stem cells.

CD47 is also constitutively upregulated on a number of cancers such as Non-Hodgkin Lymphoma (NHL), Acute myeloid leukemia (AML), breast, colon, glioblastoma, glioma, ovarian, bladder and prostate cancers. Overexpression of CD47 by tumor cells, which efficiently helps them to escape immune surveillance and killing by innate immune cells.

It has been shown that CD47 may be considered as potential target for the treatment of atherosclerosis. Because the process of atherogenesis i.e., the formation of atheromas on the wall of arteries, is associated with upregulation of CD47 which renders malignant cells resistant to programmed cell removal, or ‘efferocytosis’. This effect of efferocytosis, is reversed upon administration of CD47-blocking antibodies which normalizes the clearance of diseased vascular tissue and ameliorates atherosclerosis in multiple mouse models (Kojima Y, et al., Nature. 2016 Aug. 4; 536 (7614): 86-90). Further, it has been reported that blockade of CD47 with CD47-Fc fusion protein is effective in regulating Experimental autoimmune encephalomyelitis (EAE) in an animal model for multiple sclerosis (MS) thus provides a potential therapeutic target in preventing and treating MS (Gao Q et al., J Autoimmun. 2016 May; 69:74-85).

International publications, WO2019138367 and WO2020095256 describe inhibitors of CD47-SIRPα signaling pathway, processes for the preparation thereof, pharmaceutical compositions comprising them, their uses as therapeutic agents as monotherapy as well as combination therapy in the treatment of various disorders, particularly in the treatment of cancer and other proliferative disorders, which are incorporated herein by reference for all purposes. One of the 1,2,4-oxadiazole compounds disclosed in WO2019138367 is (((S)-1-(3-((S)-1-amino-3-carboxypropyl)-1,2,4-oxadiazol-5-yl)-4-guanidinobutyl)carbamoyl)-L-proline.

For drug development, it is typically advantageous to employ a form of the drug having desirable physicochemical properties with respect to its preparation, purification, reproducibility, stability, bioavailability, and other characteristics to prepare a suitable pharmaceutical formulation. High-purity compounds are difficult to make in pharmaceuticals due to the presence of impurities from various sources in the starting materials and/or intermediates or generation of impurities during their synthesis, formulation and/or storage. Since these impurities can affect drug efficacy, safety, and stability, necessitating high levels of purity to ensure the quality of the final product, the drug regulatory authorities have established stringent quality control measures for pharmaceutical compounds and compositions thereof.

### OBJECT OF THE INVENTION

It is an object of the present disclosure to provide a highly pure calcium salt of a CD47 SIRP-alpha inhibitor of compound of formula (I)

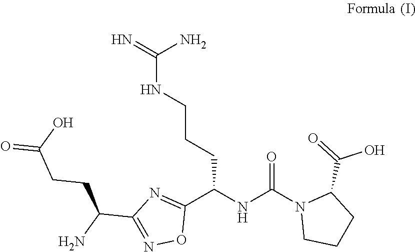

which is substantially free from impurities. More particularly, the calcium salt of the CD47 SIRP-alpha inhibitor of compound of formula (I) is substantially free from at least one impurity selected from A-1, A-2 and A-3. Such a highly pure compound not only possesses the desired pharmacological properties, but is also suitable for preparing pharmaceutical formulations and dosage forms that are safe, effective, and high-quality drug products meeting the stringent regulatory requirements.

### SUMMARY OF THE INVENTION

Compound of formula (I) is also known as (((S)-1-(3-((S)-1-amino-3-carboxypropyl)-1,2,4-oxadiazol-5-yl)-4-guanidinobutyl)carbamoyl)-L-proline. In one aspect, the present disclosure provides a highly pure calcium salt of compound of formula (I):

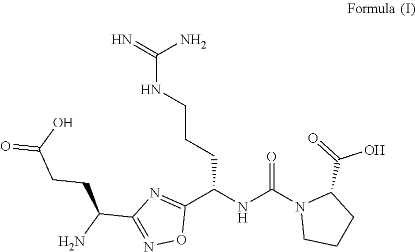

which is substantially free from impurities.

In one embodiment, the compound of formula (I) or the calcium salt of compound of formula (I) is an enantiomerically pure compound. In a preferred embodiment, the compound of formula (I) is LLL-isomer.

In some embodiments, the compound of formula (I) is enantiomerically pure or chiral pure, preferably the compound of formula (I) is LLL-isomer.

In some embodiments, the calcium salt of compound of formula (I), is substantially free from at least one impurity selected from A-1, A-2 and A-3:

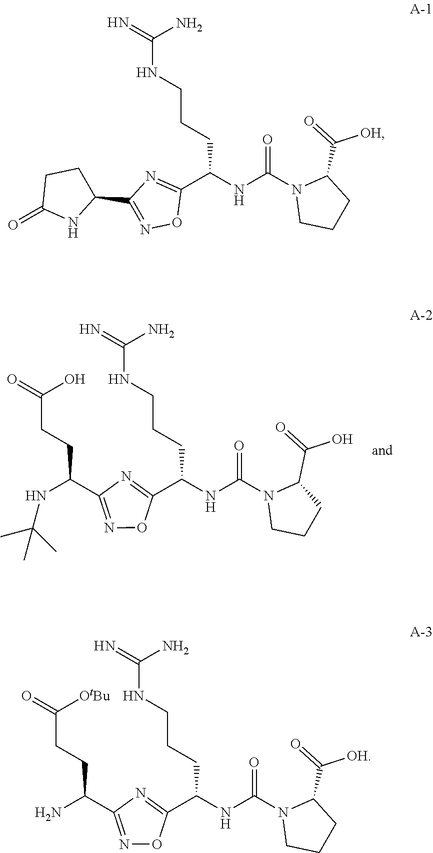 [SMILES: N=C(N)NCCC[C@H](NC(=O)N1CCC[C@H]1C(=O)O)c1nc([C@@H]2CCC(=O)N2)no1 ; CC(C)(C)N[C@@H](CCC(=O)O)c1noc([C@H](CCCNC(=N)N)NC(=O)N2CCC[C@H]2C(=O)O)n1 ; CC(C)(C)OC(=O)CC[C@H](N)c1noc([C@H](CCCNC(=N)N)NC(=O)N2CCC[C@H]2C(=O)O)n1]

The highly pure calcium salt of compound of formula (I), exhibits surprisingly good physicochemical properties such as greater stability, purity control, and better suitability for pharmaceutical development to meet the stringent regulatory requirements.

The present disclosure further relates to a pharmaceutical composition comprising the said calcium salt of compound of formula (I) as described herein.

In one aspect, the pharmaceutical composition is substantially free from at least one impurity selected from A-1, A-2 and A-3. In a preferred embodiment, the pharmaceutical composition is substantially free from impurities A-1 and A-2, or A-1 and A3, or A-2 and A-3. In another preferred embodiment, the pharmaceutical composition is substantially free from impurities A-1, A-2 and A-3.

In a most preferred embodiment, the pharmaceutical composition is substantially free from impurities A-1, A-2 and A-3 and compound of formula (I) is an LLL-isomer.

The disclosure herein encompasses any polymorphs, co-crystals, anhydrous, and amorphous forms of the said highly pure calcium salt of compound of formula (I).

In one aspect, the present disclosure provides a calcium salt of compound of formula (I):

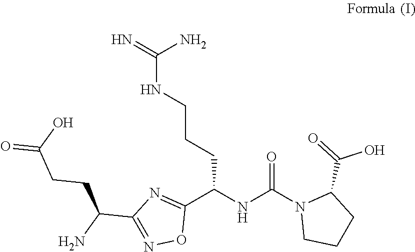

comprising at least one impurity selected from A-1, A-2 and A-3:

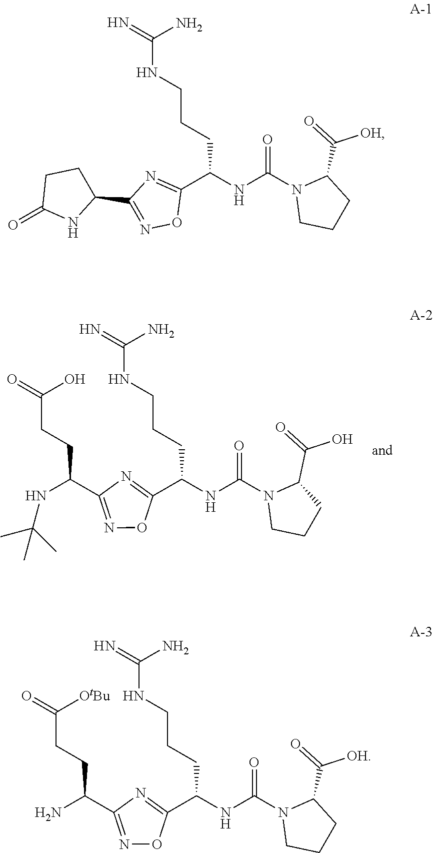 [SMILES: N=C(N)NCCC[C@H](NC(=O)N1CCC[C@H]1C(=O)O)c1nc([C@@H]2CCC(=O)N2)no1 ; CC(C)(C)N[C@@H](CCC(=O)O)c1noc([C@H](CCCNC(=N)N)NC(=O)N2CCC[C@H]2C(=O)O)n1 ; CC(C)(C)OC(=O)CC[C@H](N)c1noc([C@H](CCCNC(=N)N)NC(=O)N2CCC[C@H]2C(=O)O)n1]

In one embodiment, the calcium salt of the compound of formula (I) has a purity of at least about 93%, preferably at least about 97%, more preferably at least about 99%, or about 97.5% to 98.76% as measured by HPLC (% area).

In one embodiment, the calcium salt of the compound of formula (I) has a purity of at least about 93%, preferably at least about 97%, more preferably at least about 99.9%, or about 97.5% to 99.9% as measured by HPLC (% area).

In one aspect, the impurities A-1, A-2 and A-3 are present in a combined amount of not more than about 3%, not more than about 2%, or not more than about 1% as measured by HPLC (% area).

In one aspect, the present disclosure provides a pharmaceutical composition comprising the calcium salt of compound of formula (I), or a polymorph, co-crystal, anhydrous, or an amorphous form thereof and one or more pharmaceutically acceptable excipients.

In another aspect, the present invention provides a pharmaceutical composition comprising the calcium salt of compound of formula (I), or a polymorph, co-crystal, anhydrous, or an amorphous form thereof free from at least one impurity selected from A-1, A-2 and A-3, and one or more pharmaceutically acceptable excipients. In a preferred embodiment, the compound of formula (I) is an LLL-isomer.

In one aspect, the present disclosure provides a tablet composition comprising the calcium salt of compound of formula (I) in accordance with the present disclosure.

In one aspect, the present disclosure provides the calcium salt of compound of formula (I) or the composition according to the disclosure for use in treatment of cancer. In a preferred embodiment according to the disclosure, the highly pure compound of formula (I) for use in the treatment of cancer is an enantiomerically pure compound, preferably an LLL-isomer.

In one aspect, the present disclosure provides a method of treating cancer comprising administering a therapeutically effective amount of the said calcium salt of compound of formula (I) according to the disclosure, or the composition according to the disclosure to a patient in need thereof.

In one aspect, the present disclosure provides a use of the compound according to the disclosure, or the composition according to the disclosure, in the manufacture of a medicament for treatment of cancer.

### BRIEF DESCRIPTION OF THE DRAWINGS

FIG. 1 shows a 1H NMR spectrum of impurity A-1.

FIG. 2 shows a 1H NMR spectrum of LDL isomer impurity.

FIG. 3 shows a Scheme of Preparation of the calcium salt of compound of formula (I).

### DETAILED DESCRIPTION OF THE INVENTION

As used in the present specification, the following words and phrases are generally intended to have the meanings as set forth below, except to the extent that the context in which they are used indicates otherwise.

As used herein, the compound ‘(((S)-1-(3-((S)-1-amino-3-carboxypropyl)-1,2,4-oxadiazol-5-yl)-4-guanidinobutyl)carbamoyl)-L-proline’ or ‘formula (I) free acid form’ is the compound having the following formula:

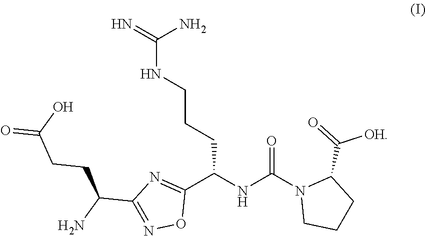

As used herein, the terms “calcium salt of compound of formula (I)”, “calcium salt of formula (I)”, “compound of formula (I) calcium salt”, “compound of formula (I) calcium”, “compound (I) Ca salt” and “pharmaceutically acceptable salt of compound of formula (I) wherein the salt is calcium” are interchangeable and refer to one or more calcium ions with an ionic bond with the compound of formula (I).

As used herein, the term “pharmaceutically acceptable” refers to those compounds, materials, compositions, and/or dosage forms which are, within the scope of sound medical judgement, suitable for use in contact with the tissues of human beings and animals without excessive toxicity, irritation, allergic response, immunogenicity or other problem or complication, commensurate with a reasonable benefit/risk ratio.

As used herein, the phrase “pharmaceutically acceptable carrier or excipient” refers to a pharmaceutically acceptable material, composition, or vehicle, such as a liquid or solid filler, diluent, solvent, or encapsulating material. Excipients or carriers are generally safe, non-toxic and neither biologically nor otherwise undesirable and include excipients or carriers that are acceptable for veterinary use as well as human pharmaceutical use.

As used herein, the singular forms “a,” “an,” and “the” include plural reference unless the context clearly dictates otherwise.

In general, the nomenclature used in this application is based on naming conventions adopted by the International Union of Pure and Applied Chemistry (IUPAC). Chemical structures shown herein were prepared using CHEMDRAW®. Any open valency appearing on a carbon, oxygen, or nitrogen atom in the structures herein indicates the presence of a hydrogen atom.

“Pharmaceutical composition” refers to a therapeutically active compound and at least one pharmaceutically acceptable carrier or excipient, with which the compound is administered to a patient.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans.

As used herein, “therapeutically effective amount” refers to an amount that is sufficient to effect treatment, when administered to a mammal in need of such treatment. The therapeutically effective amount will vary depending upon the subject being treated, the weight and age of the subject, the severity of the disease condition, the manner of administration and the like, which can readily be determined by one of ordinary skill in the art.

As used herein, the terms “treating” or “treatment” refer to inhibiting the disease; for example, inhibiting the disease, condition, syndrome or disorder, inhibiting the progression or worsening of signs and symptoms of a disease, condition, syndrome or disorder in an individual who is experiencing or displaying the pathology or symptomatology of the disease, condition or disorder, arresting and/or slowing down further development of the pathology and/or symptoms; and (2) ameliorating the disease; for example, ameliorating or abrogating a disease, condition or disorder in an individual who is experiencing or displaying the pathology or symptomatology of the disease, condition or disorder such as decreasing or reversing the severity of disease.

As used herein, “crystalline” or “crystalline form” is meant to refer to a certain lattice configuration of a crystalline substance. Different crystalline forms of the same substance typically have different crystalline lattices (e.g., unit cells) which may contribute to different physical properties that are characteristic of each of the crystalline forms. In some instances, different lattice configurations have different water or solvent content.

As used herein, “cocrystal” refers to single phase crystalline material comprising at least two non-volatile components in the same crystal lattice in a specific stoichiometric (molar) ratio, where the arrangement in the crystal lattice is not based on ionic bonds (as with salts) or covalent interactions and at least two of the components are solids at room temperature. Cocrystals can encompass hydrates, solvates and clathrates.

Each embodiment is provided by way of explanation of the disclosure, and not by way of limitation of the disclosure. In fact, it will be apparent to those skilled in the art that various modifications and variations can be made to the compounds, compositions, and methods described herein without departing from the scope or spirit of the disclosure. For instance, features illustrated or described as part of some embodiments can be applied to another embodiment to yield a still further embodiment. Thus, it is intended that the present disclosure include such modifications and variations and their equivalents. Other objects, features, and aspects of the present disclosure are disclosed in, or are obvious from, the following detailed description. It is to be understood by one of ordinary skill in the art that the present discussion is a description of exemplary embodiments only and is not to be construed as limiting the broader aspects of the present disclosure.

As used herein, the term “comprise” or “comprising” is generally used in the sense of include, that is to say permitting the presence of one or more additional (unspecified) features or components.

As used herein, the term “including” as well as other forms, such as “include”, “includes,” and “included,” is not limiting.

The terms “highly pure” or “high purity” or “substantially pure” or “substantially free of impurity/impurities”, as used herein have substantially the same meaning and can be used interchangeably. Such terms refer to calcium salt of compound of formula (I) according to the disclosure and/or the pharmaceutical composition comprising said calcium salt of compound of formula (I). When any of these terms are used in conjunction with calcium salt of compound of formula (I) or the pharmaceutical composition comprising said calcium salt of compound of formula (I), this would mean that said calcium salt of compound of formula (I) as such, or as comprised in the pharmaceutical composition is having purity of at least about 93.0%, at least about 94.0%, at least about 95.0%, at least about 96.0%, at least about 97.0%, at least about 98.0%, at least about 99.0%, at least about 99.5%, or at least about 99.9%, as measured by HPLC (% area). Preferably, at least about 97.0%, more preferably at least about 99.0%, as measured by HPLC (% area).

In some embodiments, when any of these terms are used in conjunction with calcium salt of compound of formula (I) or the pharmaceutical composition comprising said calcium salt of compound of formula (I), this would mean that said calcium salt of compound of formula (I) as such, or as comprised in the pharmaceutical composition, contains total impurities not more than about 7.0% or not more than about 1.0%, or not more than about 6.0%, not more than about 5.0%, not more than about 4.0%, not more than about 4.0%, not more than about 3.0%, not more than about 2.0%, as measured by HPLC (% area). Preferably, said calcium salt of compound of formula (I) contains any individual identified or any individual unidentified impurity not more than about 1.5%, not more than about 1.0%, not more than about 0.5%, not more than about 0.1%, not more than about 0.05%, as measured by HPLC (% area). The term may also encompass compliance with pharmacopoeia standards or regulatory guidelines for purity, where applicable.

As used herein, the term “not more than” refers to a quantity that is equal to or less than the specified numerical value.

As used herein, the term “at least” refers to a quantity that is equal to or greater than the specified numerical value.

As used herein, the term “less than” refers to a quantity that is strictly below the specified numerical value, excluding the stated value itself.

In one aspect, the present disclosure provides a highly pure calcium salt of compound of formula (I). In preferred aspects, the highly pure calcium salt of compound of formula (I) is an enantiomerically pure LLL-isomer.

In one aspect, the present disclosure provides a calcium salt of compound of formula (I)

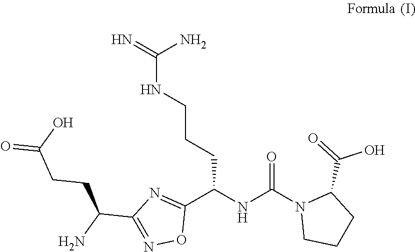

wherein the calcium salt of the compound is substantially free from impurities. In a preferred embodiment, the calcium salt of the compound of formula (I) is LLL-isomer. In a more preferred embodiment, the calcium salt of compound of formula (I) is an enantiomerically pure LLL-isomer which is free from other isomeric impurities.

In some embodiments, the compound of formula (I) is enantiomerically pure or chiral pure, preferably the compound of formula (I) is LLL-isomer.

In one aspect, the present disclosure provides a calcium salt of compound of formula (I)

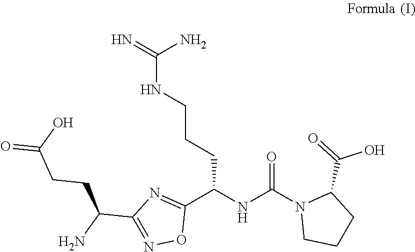

wherein the calcium salt of the compound is substantially free from at least one impurity selected from A-1, A-2 and A-3:

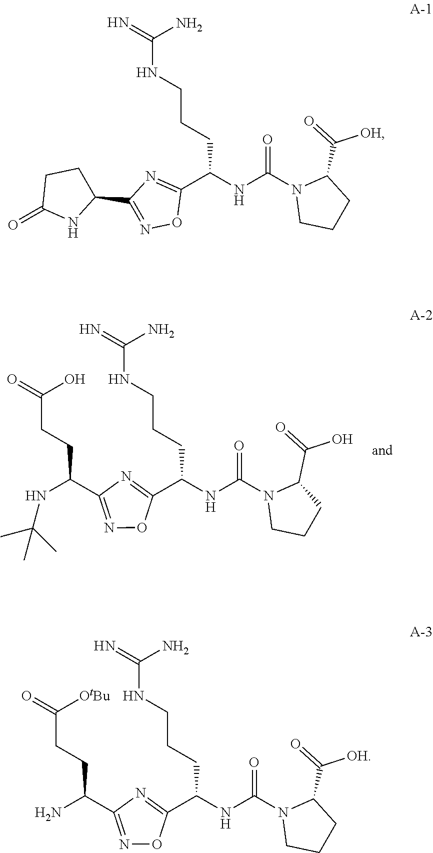 [SMILES: N=C(N)NCCC[C@H](NC(=O)N1CCC[C@H]1C(=O)O)c1nc([C@@H]2CCC(=O)N2)no1 ; CC(C)(C)N[C@@H](CCC(=O)O)c1noc([C@H](CCCNC(=N)N)NC(=O)N2CCC[C@H]2C(=O)O)n1 ; CC(C)(C)OC(=O)CC[C@H](N)c1noc([C@H](CCCNC(=N)N)NC(=O)N2CCC[C@H]2C(=O)O)n1]

In one aspect, the calcium salt of the compound of formula (I) has a purity of at least about 93%, preferably at least about 97%, more preferably at least about 99%, or about 97.5% to 98.76% as measured by HPLC (% area).

In some embodiments, the calcium salt of compound of formula (I) has a purity of at least about 93%, preferably at least about 97%, or about 97.5% to 99.9%, more preferably at least about 99.9%, as measured by HPLC (% area).

In some embodiments, the calcium salt of compound of formula (I) has a purity of at least about 93.5%, 93.6%, 93.7%, 93.8% or 93.9% as measured by HPLC (% area).

In some embodiments, the calcium salt of compound of formula (I) has a purity of at least about 93.5% as measured by HPLC (% area).

In some embodiments, the calcium salt of compound of formula (I) has a purity of at least about 93.6% as measured by HPLC (% area).

In some embodiments, the calcium salt of compound of formula (I) has a purity of at least about 97.5%, 97.6%, 98.7%, 97.8%, or 97.9% as measured by HPLC (% area).

In some embodiments, the calcium salt of compound of formula (I) has a purity of at least about 98.1%, 98.2%, 98.3%, 98.4%, 98.5%, 98.6%, 98.7%, 98.8% or 98.9% as measured by HPLC (% area).

In some embodiments, the calcium salt of compound of formula (I) has a purity of at least about 98.3% as measured by HPLC (% area).

In some embodiments, the calcium salt of compound of formula (I) has a purity of at least about 98.6% as measured by HPLC (% area).

In some embodiments, the calcium salt of compound of formula (I) has a purity of at least about 98.7% as measured by HPLC (% area).

In some embodiments, the calcium salt of compound of formula (I) has a purity of at least about 98.72% as measured by HPLC (% area).

In some embodiments, the calcium salt of compound of formula (I) has a purity of at least about 98.76% as measured by HPLC (% area).

In some embodiments, the calcium salt of compound of formula (I) has a purity of at least about 99.1%, 99.2%, 99.3%, 99.4%, 99.5%, 99.6%, 99.7%, 99.8% or 99.9% as measured by HPLC (% area).

In some embodiments, the calcium salt of compound of formula (I) has a purity of at least about 99.1% as measured by HPLC (% area).

In some embodiments, the calcium salt of compound of formula (I) has a purity of at least about 99.2% as measured by HPLC (% area).

In some embodiments, the calcium salt of compound of formula (I) has a purity of at least about 99.3% as measured by HPLC (% area).

In some embodiments, the calcium salt of compound of formula (I) has a purity of at least about 99.6% as measured by HPLC (% area).

In some embodiments, the calcium salt of compound of formula (I) has a purity of at least about 99.68% as measured by HPLC (% area).

In some embodiments, the impurities A-1, A-2 and A-3 are present in a combined amount of not more than about 3%, not more than about 2%, or not more than about 1% as measured by HPLC (% area).

In some embodiments, at least one impurity selected from A-1, A-2 and A-3 is not more than about 0.017% as measured by HPLC (% area).

In some embodiments, the impurity A-1 is not more than about 1.30% as measured by HPLC (% area).

In some embodiments, the impurity A-1 is in the range of about 0.017% to 1.30% as measured by HPLC (% area).

In some embodiments, the impurity A-2 is not more than about 0.60% as measured by HPLC (% area).

In some embodiments, the impurity A-2 is in the range of about 0.017% to 0.60% as measured by HPLC (% area).

In some embodiments, the impurity A-3 is not more than about 0.60% as measured by HPLC (% area).

In some embodiments, the impurity A-3 is in the range of about 0.017% to 0.60% as measured by HPLC (% area).

Impurities may form or be otherwise introduced during the synthesis or storage of the calcium salt of the compound of formula (I). Such impurities may include, but are not limited to, synthetic intermediates, degradation products, inorganic materials (e.g., metals used in catalytic reactions), and residual solvents. Reducing and/or eliminating such impurities is generally advantageous, and minimizing the amounts of genotoxic impurities is particularly important for reducing health risks upon administration to patients. The genotoxicity of an impurity or other material can be assessed by methods such the Ames test (bacterial reverse mutation assay; see, Mortelmans et al. Mutat Res 2000; 455: 29e60) or a micronucleus assay (OECD (2016), “Test No. 474: Mammalian Erythrocyte Micronucleus Test,” OECD Guidelines for the Testing of Chemicals, Section 4, OECD Publishing, Paris).

In one embodiment, upon storage of the calcium salt of the compound of formula (I) at 2-8 °C, the impurity A-1 remains less than about 3%, preferably less than about 1.3% as measured by HPLC (% area) for a period of 12 months, preferably 18 months.

In some embodiments, upon storage of the calcium salt of the compound of formula (I) at 2-8 °C, the impurity A-1 remains less than about 3%, preferably less than about 1.3% as measured by HPLC (% area) for a period of 12 months, preferably 18 months, more preferably 24 months.

In some embodiments, upon storage of the calcium salt of the compound of formula (I) at 25±2 °C/60%±5% RH, the impurity A-1 remains less than about 3%, preferably less than about 1.3% as measured by HPLC (% area) for a period of 12 months, preferably 18 months.

In some embodiments, upon storage of the calcium salt of the compound of formula (I) at 25±2 °C/60%±5% RH, the impurity A-1 remains less than about 3%, preferably less than about 1.3% as measured by HPLC (% area) for a period of 12 months, preferably 18 months, more preferably 24 months.

In some embodiments, upon storage of the calcium salt of the compound of formula (I) at 2-8 °C, the impurity A-2 remains less than about 1%, preferably less than about 0.60% as measured by HPLC (% area) for a period of 12 months, preferably 18 months.

In some embodiments, upon storage of the calcium salt of the compound of formula (I) at 2-8 °C, the impurity A-2 remains less than about 1%, preferably less than about 1.3% as measured by HPLC (% area) for a period of 12 months, preferably 18 months, more preferably 24 months.

In some embodiments, upon storage of the calcium salt of the compound of formula (I) at 25±2 °C/60%±5% RH, the impurity A-2 remains less than about 1%, preferably less than about 0.60% as measured by HPLC (% area) for a period of 12 months, preferably 18 months.

In some embodiments, upon storage of the calcium salt of the compound of formula (I) at 25±2 °C/60%±5% RH, the impurity A-2 remains less than about 1%, preferably less than about 0.60% as measured by HPLC (% area) for a period of 12 months, preferably 18 months, more preferably 24 months.

In some embodiments, upon storage of the calcium salt of the compound of formula (I) at 2-8 °C, the impurity A-3 remains less than about 1%, preferably less than about 0.60% as measured by HPLC (% area) for a period of 12 months, preferably 18 months.

In some embodiments, upon storage of the calcium salt of the compound of formula (I) at 2-8 °C, the impurity A-3 remains less than about 1%, preferably less than about 0.60% as measured by HPLC (% area) for a period of 12 months, preferably 18 months, more preferably 24 months.

In some embodiments, upon storage of the calcium salt of the compound of formula (I) at 25±2 °C/60%±5% RH, the impurity A-3 remains less than about 1%, preferably less than about 0.60% as measured by HPLC (% area) for a period of 12 months, preferably 18 months.

In some embodiments, upon storage of the calcium salt of the compound of formula (I) at 25±2 °C/60%±5% RH, the impurity A-3 remains less than about 1%, preferably less than about 0.60% as measured by HPLC (% area) for a period of 12 months, preferably 18 months, more preferably 24 months.

In some embodiments, upon storage of the calcium salt of the compound of formula (I) at 2-8 °C, the impurities A1 and A2 remain less than about 3%, preferably less than about 1.3% as measured by HPLC (% area) for a period of 12 months, preferably 18 months, more preferably 24 months.

In some embodiments, upon storage of the calcium salt of the compound of formula (I) at 2-8 °C, the impurities A2 and A3 remain less than about 1%, preferably less than about 0.6% as measured by HPLC (% area) for a period of 12 months, preferably 18 months, more preferably 24 months.

In some embodiments, upon storage of the calcium salt of the compound of formula (I) at 2-8 °C, the impurities A1, A2 and A3 remain less than about 3%, preferably less than about 1.3% as measured by HPLC (% area) for a period of 12 months, preferably 18 months, more preferably 24 months.

In some embodiments, upon storage of the calcium salt of the compound of formula (I) at 25±2 °C/60%±5% RH, the impurities A1 and A2 remain less than about 3%, preferably less than about 1.3% as measured by HPLC (% area) for a period of 12 months, preferably 18 months, more preferably 24 months.

In some embodiments, upon storage of the calcium salt of the compound of formula (I) at 25±2 °C/60%±5% RH, the impurities A2 and A3 remain less than about 1%, preferably less than about 0.6% as measured by HPLC (% area) for a period of 12 months, preferably 18 months, more preferably 24 months.

In some embodiments, upon storage of the calcium salt of the compound of formula (I) at 25±2 °C/60%±5% RH, the impurities A1, A2 and A3 remain less than about 3%, preferably less than about 1.3% as measured by HPLC (% area) for a period of 12 months, preferably 18 months, more preferably 24 months.

In a preferred embodiment, the calcium salt of compound of formula (I) according to above embodiments, wherein the compound of formula (I) is an enantiomerically pure LLL-isomer.

In some embodiments, the calcium salt of compound of formula (I) is substantially free from at least one of the following impurities as measured by HPLC (% area):
a. not more than about 0.50% of impurity at RRT 0.25;
b. not more than about 0.50% of impurity at RRT 0.86;
c. not more than about 0.20% of impurity at RRT 0.56;
d. not more than about 0.30% of impurity at RRT 0.68;
e. not more than about 0.15% of impurity at RRT 1.07;
f. not more than about 0.20% of impurity at RRT 1.21;
g. not more than about 0.20% of impurity at RRT 1.42; or
h. not more than about 0.10% of other single unspecified impurity;
   or
a. not more than about 0.50% of impurity at RRT 0.25;
b. not more than about 0.50% of impurity at RRT 0.86;
c. not more than about 0.50% of any other single unspecified impurity, preferably selected from: impurity at RRT 0.56, impurity at RRT 0.68, impurity at RRT 1.07, impurity at RRT 1.21 and impurity at RRT 1.42;
   or
a. not more than about 0.08%, preferably not more than about 0.07% of impurity at RRT 0.25;
b. not more than about 0.38%, preferably not more than about 0.24% of impurity at RRT 0.86; or
c. not more than about 0.13%, preferably not more than about 0.10% of any other single unspecified impurity selected from: impurity at RRT 0.56, impurity at RRT 0.68, impurity at RRT 1.07, impurity at RRT 1.21 and impurity at RRT 1.42.

In some embodiments, the calcium salt of compound of formula (I) has a chiral purity of about 94.6% to 99.2%, preferably at least about 98%, more preferably at least about 99% as measured by chiral HPLC (% area).

In some embodiments, the calcium salt of compound of formula (I) is substantially free from at least one of the following impurities as measured by chiral HPLC (% area):
a. LDL isomer impurity (RRT 0.86) of not more than about 1.90%, preferably not more than about 1.50%, more preferably not more than about 0.78%, even more preferably in the range of about 0.78% to 1.90%;
b. all other isomers not more than about 3.50%, preferably about not more than 1.00%, more preferably not more than about 0.02%, even more preferably in the range of about 0.02% to 3.50%, wherein the isomers are selected from one or more of: LLL isomer impurity, DLD isomer impurity, DDL isomer impurity, DLL isomer impurity, DDD isomer impurity, LLD isomer impurity and LDD isomer impurity.

In some embodiments, the calcium salt of compound of formula (I) LLL-isomer is substantially free from at least one of the following impurities as measured by chiral HPLC (% area):
a. LDL isomer impurity (RRT 0.86) of not more than about 1.90%, preferably not more than about 1.50%, more preferably not more than about 0.78%, even more preferably in the range of about 0.78% to 1.90%;
b. all other isomers not more than about 3.50%, preferably about not more than 1.00%, more preferably not more than about 0.02%, even more preferably in the range of about 0.02% to 3.50%, wherein the isomers are selected from one or more of: DLD isomer impurity, DDL isomer impurity, DLL isomer impurity, DDD isomer impurity, LLD isomer impurity and LDD isomer impurity.

In some embodiments, the calcium salt of compound of formula (I) LLL-isomer is substantially free from one of the following impurities as measured by chiral HPLC (% area):
a. LDL isomer impurity (RRT 0.86) of not more than about 1.90%, preferably not more than about 1.50%, more preferably not more than about 0.78%, even more preferably in the range of about 0.78% to 1.90%; and
b. all other isomers not more than about 3.50%, preferably about not more than 1.00%, more preferably not more than about 0.02%, even more preferably in the range of about 0.02% to 3.50%, wherein the isomers are selected from one or more of: DLD isomer impurity, DDL isomer impurity, DLL isomer impurity, DDD isomer impurity, LLD isomer impurity and LDD isomer impurity.

In some embodiments, the calcium salt of compound of formula (I) is substantially free from at least one of the following impurities as measured by Inductively coupled plasma mass spectrometry (ICP-MS):
a. not more than about 10 ppm of Vanadium;
b. not more than about 5 ppm of Cobalt;
c. not more than about 20 ppm of Nickel;
d. not more than about 1.5 ppm of Arsenic;
e. not more than about 0.5 ppm of Cadmium;
f. not more than about 3 ppm of Mercury;
g. not more than about 0.5 ppm of Lead; or
h. not more than about 10 ppm of Palladium.

In one aspect, the present disclosure provides a pharmaceutical composition comprising the LLL-isomer of calcium salt of compound of formula (I), or a polymorph, co-crystal, anhydrous, or an amorphous form thereof free from at least one impurity selected from A-1, A-2 and A-3, and one or more pharmaceutically acceptable excipients.

In one aspect, the present disclosure provides a calcium salt of compound of formula (I),

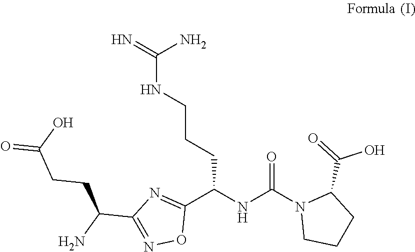

comprising at least one impurity selected from A-1, A-2 and A-3:

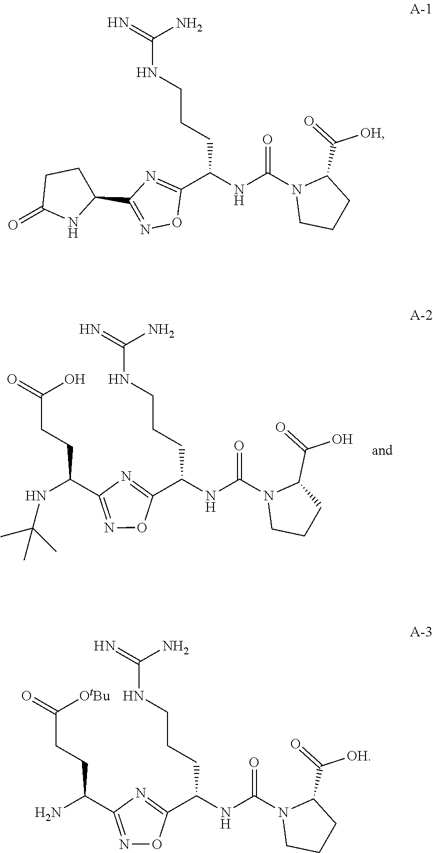 [SMILES: N=C(N)NCCC[C@H](NC(=O)N1CCC[C@H]1C(=O)O)c1nc([C@@H]2CCC(=O)N2)no1 ; CC(C)(C)N[C@@H](CCC(=O)O)c1noc([C@H](CCCNC(=N)N)NC(=O)N2CCC[C@H]2C(=O)O)n1 ; CC(C)(C)OC(=O)CC[C@H](N)c1noc([C@H](CCCNC(=N)N)NC(=O)N2CCC[C@H]2C(=O)O)n1]

In another aspect, the calcium salt of compound of formula (I) comprises impurity A-1.

In another aspect, the calcium salt of compound of formula (I) comprises impurity A-1 in the range of about 0.017% to 1.30% as measured by HPLC (% area).

In another aspect, the calcium salt of compound of formula (I) comprises impurity A-2.

In another aspect, the calcium salt of compound of formula (I) comprises impurity A-2 in the range of about 0.017% to 0.60% as measured by HPLC (% area).

In another aspect, the calcium salt of compound of formula (I) comprises impurity A-3.

In another aspect, the calcium salt of compound of formula (I) comprises impurity A-3 in the range of about 0.017% to 0.60% as measured by HPLC (% area).

In some embodiments, the calcium salt of the compound of formula (I) has a purity of at least about 93%, preferably at least about 97%, about 97.5% to 98.76%, more preferably at least about 99% as measured by HPLC (% area).

In some embodiments, the calcium salt of the compound of formula (I) has a purity of at least about 93%, preferably at least about 97%, about 97.5% to 99.9%, more preferably at least about 99.9% as measured by HPLC (% area).

In some embodiments, the calcium salt of compound of formula (I) comprises impurities A1, A2 and A3 in the range of about 0.017% to 1.30% as measured by HPLC (% area).

In some embodiments, the calcium salt of the compound of formula (I) comprises impurities A1, A2 and A3 and wherein the calcium salt of the compound of formula (I) has a purity of at least about 93%, preferably at least about 97%, about 97.5% to 99.9%, more preferably at least about 99.9% as measured by HPLC (% area).

In preferred embodiments, the calcium salt of compound of formula (I) according to one or more of above embodiments, wherein the compound of formula (I) is an LLL-isomer.

In some embodiments, the compound of formula (I) is enantiomerically pure or chiral pure, preferably the compound of formula (I) is LLL-isomer.

In some embodiments, the present disclosure provides a pharmaceutical composition comprising the calcium salt of compound of formula (I), or a polymorph, co-crystal, anhydrous, or amorphous form thereof comprising at least one impurity selected from A-1, A-2 and A-3, and one or more pharmaceutically acceptable excipients. In preferred embodiments, the compound of formula (I) is an LLL-isomer.

In some embodiments, the present disclosure provides a pharmaceutical composition comprising the calcium salt of compound of formula (I), or a polymorph, co-crystal, anhydrous, or amorphous form thereof comprising impurity A-1, and one or more pharmaceutically acceptable excipients. In preferred embodiments, the compound of formula (I) is an LLL-isomer.

In some embodiments, the present disclosure provides a pharmaceutical composition comprising the calcium salt of compound of formula (I), or a polymorph, co-crystal, anhydrous, or amorphous form thereof comprising impurity A-2, and one or more pharmaceutically acceptable excipients. In preferred embodiments, the compound of formula (I) is an LLL-isomer.

In some embodiments, the present disclosure provides a pharmaceutical composition comprising the calcium salt of compound of formula (I), or a polymorph, co-crystal, anhydrous, or amorphous form thereof comprising impurity A-3, and one or more pharmaceutically acceptable excipients. In preferred embodiments, the compound of formula (I) is an LLL-isomer.

In some embodiments, upon storage of the calcium salt of the compound of formula (I) at 2-8 °C, the calcium salt of the compound remains substantially free of the impurities for a period of 12 months, preferably 18 months.

In some embodiments, upon storage of the calcium salt of the compound of formula (I) at 2-8 °C, the calcium salt of the compound remains substantially free of the impurities for a period of 12 months, preferably 18 months, more preferably 24 months. In preferred embodiments, the compound of formula (I) is an LLL-isomer.

In some preferred embodiments:
(i) the purity of calcium salt of compound of formula (I) remains not less than 97%, preferably not less than 99% as measured by HPLC (% area);
(ii) remains substantially free from at least one impurity selected from A-1, A-2 and A-3 as measured by HPLC (% area);
(iii) at least one impurity selected from A-1, A-2 and A-3 is not more than about 0.017%; preferably impurity A-1 is not more than about 1.30%, or impurity A-1 is in the range of about 0.017% to 1.30%; further preferably impurity A-2 is not more than about 0.60%, or impurity A-2 is in the range of about 0.017% to 0.60%; even further preferably impurity A-3 is not more than about 0.60%, or impurity A-3 is in the range of about 0.017% to 0.60% as measured by HPLC (% area);
(iv) not more than about 0.50% of impurity at RRT 0.25; not more than about 0.50% of impurity at RRT 0.86; or not more than about 0.50% of other single unspecified impurity, preferably selected from: impurity at RRT 0.56, impurity at RRT 0.68, impurity at RRT 1.07, impurity at RRT 1.21 and impurity at RRT 1.42, as measured by HPLC (% area);
(v) the chiral purity of about 94.6% to 99.2%, preferably at least about 98%, more preferably at least about 99% as measured by chiral HPLC (% area); and/or,
(vi) LDL isomer impurity (RRT 0.86) of not more than about 1.90%, preferably not more than about 1.50%, more preferably not more than about 0.78% %, even more preferably in the range of about 0.78% to 1.90%; or all other isomers not more than about 3.50%, preferably not more than about 1.00%, preferably not more than about 0.02%, even more preferably in the range of about 0.02% to 3.50%, wherein all other isomers are preferably selected from one or more of: DLD isomer impurity, DDL isomer impurity, DLL isomer impurity, DDD isomer impurity, LLD isomer impurity and LDD isomer impurity, as measured by chiral HPLC (% area).

In some embodiments, upon storage of the calcium salt of the compound of formula (I) at 25±2 °C/60±5% RH, the calcium salt of the compound remains substantially free of the impurities for a period of 12 months, preferably 18 months. In preferred embodiments, the compound of formula (I) is an LLL-isomer.

In some embodiments, upon storage of the calcium salt of the compound of formula (I) at 25±2 °C/60±5% RH, the calcium salt of the compound remains substantially free of the impurities for a period of 12 months, preferably 18 months, more preferably 24 months. In preferred embodiments, the compound of formula (I) is an LLL-isomer.

In some preferred embodiments:
(i) the purity of calcium salt of compound of formula (I) remains not less than 97%, preferably not less than 99% as measured by HPLC (% area);
(ii) remains substantially free from at least one impurity selected from A-1, A-2 and A-3 as measured by HPLC (% area);
(iii) at least one impurity selected from A-1, A-2 and A-3 is not more than about 0.017%; preferably impurity A-1 is not more than about 1.30%, or impurity A-1 is in the range of about 0.017% to 1.30%; further preferably impurity A-2 is not more than about 0.60%, or impurity A-2 is in the range of about 0.017% to 0.60%; even further preferably impurity A-3 is not more than about 0.60%, or impurity A-3 is in the range of about 0.017% to 0.60% as measured by HPLC (% area);
(iv) not more than about 0.50% of impurity at RRT 0.25; not more than about 0.50% of impurity at RRT 0.86; or not more than about 0.50% of other single unspecified impurity, preferably selected from: impurity at RRT 0.56, impurity at RRT 0.68, impurity at RRT 1.07, impurity at RRT 1.21 and impurity at RRT 1.42, as measured by HPLC (% area);
(v) the chiral purity of about 94.6% to 99.2%, preferably at least about 98%, more preferably at least about 99% as measured by chiral HPLC (% area); and/or,
(vi) LDL isomer impurity (RRT 0.86) of not more than about 1.90%, preferably not more than about 1.50%, more preferably not more than about 0.78% %, even more preferably in the range of about 0.78% to 1.90%; or all other isomers not more than about 3.50%, preferably not more than about 1.00%, preferably not more than about 0.02%, even more preferably in the range of about 0.02% to 3.50%, wherein all other isomers are preferably selected from one or more of: DLD isomer impurity, DDL isomer impurity, DLL isomer impurity, DDD isomer impurity, LLD isomer impurity and LDD isomer impurity, as measured by chiral HPLC (% area).

In one embodiment, the calcium salt of compound of formula (I) or the pharmaceutical composition comprising said calcium salt of compound of formula (I), upon storage at 2-8 °C for a period of 12 months, preferably 18 months, more preferably 24 months, or at 25±2 °C/60%=5% RH for a period of 12 months, preferably 18 months, more preferably 24 months, is characterized by at least one of the following:
(i) the purity of calcium salt of compound of formula (I) remains not less than 97%, preferably not less than 99% as measured by HPLC (% area);
(ii) remains substantially free from at least one impurity selected from A-1, A-2 and A-3 as measured by HPLC (% area);
(iii) at least one impurity selected from A-1, A-2 and A-3 is not more than about 0.017%; preferably impurity A-1 is not more than about 1.30%, or impurity A-1 is in the range of about 0.017% to 1.30%; further preferably impurity A-2 is less not more than about 0.60%, or impurity A-2 is in the range of about 0.017% to 0.60%; even further preferably impurity A-3 is less not more than about 0.60%, or impurity A-3 is in the range of about 0.017% to 0.60% as measured by HPLC (% area);
(iv) not more than about 0.50% of impurity at RRT 0.25; not more than about 0.50% of impurity at RRT 0.86; or not more than about 0.50% of other single unspecified impurity, preferably selected from: impurity at RRT 0.56, impurity at RRT 0.68, impurity at RRT 1.07, impurity at RRT 1.21 and impurity at RRT 1.42, as measured by HPLC (% area).
(v) the chiral purity of about 94.6% to 99.2%, preferably at least about 98%, more preferably at least about 99% as measured by chiral HPLC (% area);
(vi) LDL isomer impurity (RRT 0.86) of not more than about 1.90%, preferably not more than about 1.50%, more preferably not more than about 0.78% %, even more preferably in the range of about 0.78% to 1.90%; or all other isomers not more than about 3.50%, preferably not more than about 1.00%, preferably not more than about 0.02%, even more preferably in the range of about 0.02% to 3.50%, wherein all other isomers are preferably selected from one or more of: DLD isomer impurity, DDL isomer impurity, DLL isomer impurity, DDD isomer impurity, LLD isomer impurity and LDD isomer impurity, as measured by chiral HPLC (% area).

In one embodiment, the present disclosure provides a method of preparation of the calcium salt of compound of formula (I), the method comprising:
i. reacting compound a with ethylchloroformate and N-methylmorpholine (NMM) in presence of aq. ammonia to afford compound b,

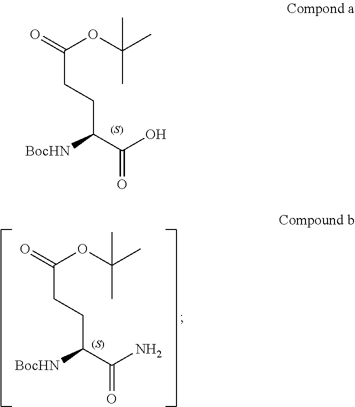

ii. reacting compound b with trifluroacetic anhydride and triethylamine to afford compound c,

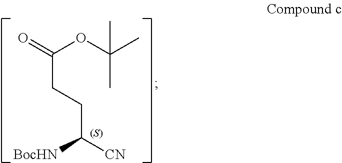

iii. reacting compound c with hydroxylamine hydrochloride, and N, N-Di-isopropyl ethylamine to afford compound d,

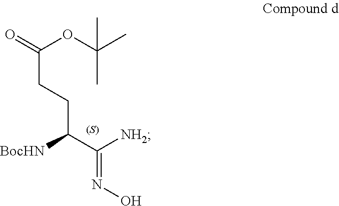

iv. reacting compound d with a compound e in presence of 1-Hydroxybenzotriazole monohydrate and N, N-Di isopropyl carbodiimide (DIC) to afford compound f,

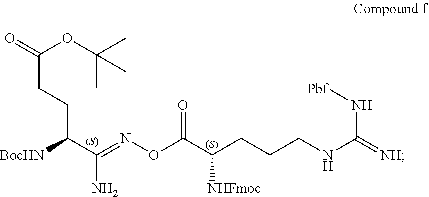

v. reacting compound f with a solvent (E) and heating the reaction mass to afford compound g,

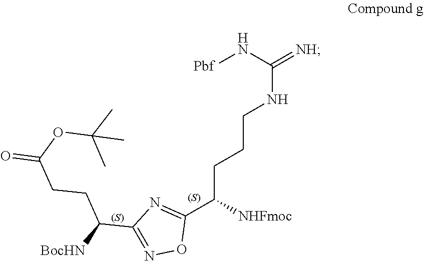

vi. reacting compound g with piperidine to afford compound h,

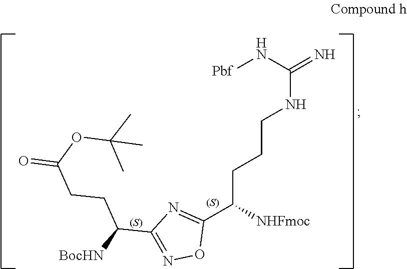

vii. reacting compound h with N,N′-Carbonyl diimidazole (CDI) followed by L-proline solution to afford compound i,

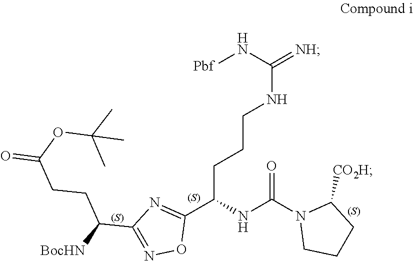

viii. deprotecting compound i with a cocktail mixture of Trifluoroacetic acid (TFA), Triisopropyl silane (TIPS) to afford the compound of formula (I),

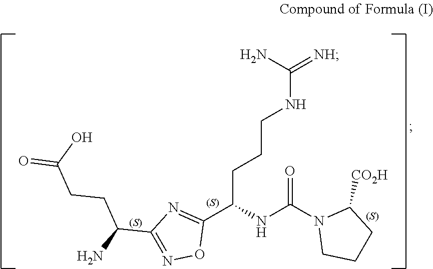

and
ix. reacting compound of formula (I) with aq. calcium carbonate to afford the calcium salt of the compound of formula (I),

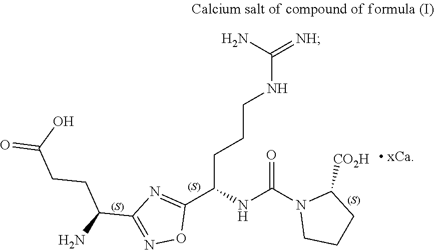

In one embodiment, the process step (i) is carried out in presence of the solvent (A) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the process step (ii) is carried out in presence of the solvent (B) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the process step (iii) is carried out in presence of the solvent (C) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the process step (iv) is carried out in presence of the solvent (D) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the process step (v) the solvent (E) is selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the process step (vi) is carried out in presence of the solvent (D) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the process step (vii) the L-proline solution is prepared in solvent (G) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, the deprotection process step (viii) is carried out in presence of the solvent (H) selected from acetone, dimethyl acetamide, dichloromethane, N,N-Dimethyl formamide, methyl tert-butyl ether, n-propyl acetate, acetonitrile, methanol, isopropyl alcohol, isopropyl acetate, isobutanol, methanol, 2-butanol, 1-butanol, n-butyl acetate, 1-pentanol, 1-propanol, chloroform, methyl acetate, isobutyl acetate, isobutanol, ethanol, water, or mixtures thereof.

In one embodiment, in the process step (iv) the compound e is an N-protected amine, wherein, the N-protecting group is selected from non-limiting examples of Pbf, Boc, Fmoc, Cbz, Bn, or Alloc.

In a preferred embodiment, the process step (i) is carried out in the presence of dichloromethane.

In a preferred embodiment, the process step (i) is carried out in the presence of NMM, ECF and dichloromethane.

In a preferred embodiment, the process step (ii) is carried out in the presence of dichloromethane.

In a preferred embodiment, the process step (ii) is carried out in the presence of trifluroacetic anhydride, triethylamine and dichloromethane.

In a preferred embodiment, the process step (iii) is carried out in the presence of methanol.

In a preferred embodiment, the process step (iii) is carried out in the presence of hydroxylamine hydrochloride, N,N-Diisopropyl ethylamine and methanol.

In a preferred embodiment, the process step (iv) is carried out in the presence of N,N-dimethyl formamide.

In a preferred embodiment, the process step (iv) is carried out in the presence of 1-hydroxybenzotriazole monohydrate, N,N-Diisopropyl carbodiimide (DIC) and N,N-dimethyl formamide.

In a preferred embodiment, the sidechain of compound e used in step (iv) is pbf-protected.

In a more preferred embodiment, in the process step (iv) the pbf-protected amine is N²-(((9H-fluoren-9-yl)methoxy)carbonyl)-N^{w}-((2,2,4,6,7-pentamethyl-2,3-dihydrobenzofuran-5-yl)sulfonyl)-L-arginine or Fmoc-Arg(Pbf)-OH.

In a preferred embodiment, the process step (v) is carried out in the presence of N,N-dimethyl formamide and dimethyl acetamide.

In a preferred embodiment, the process step (vi) is carried out in the presence of methyl tert-butyl ether (MTBE).

In a preferred embodiment, the process step (vi) is carried out in presence of piperidine and methyl tert-butyl ether (MTBE).

In a preferred embodiment, the process step (vii) is carried out in presence of N,N′-carbonyl diimidazole (CDI) and DMF followed by the addition of L-proline solution.

In a more preferred embodiment, the process step (vii) is carried out in the presence of CDI, DMF, L-proline solution, and 4-dimethylamino pyridine (DMAP).

In a more preferred embodiment, the process step (vii), wherein the L-proline solution is a mixture of L-proline and DIPEA in methanol.

In a preferred embodiment, the deprotection process step (viii) is carried out in the presence of Trifluoroacetic acid and water.

In a preferred embodiment, the process step (viii) is carried out in presence of a cocktail mixture of Trifluoroacetic acid (TFA), Triisopropyl silane (TIPS), and water.

In a more preferred embodiment, the process step (viii) is carried out by stirring the reaction mixture at 5±5 °C followed by stirring at 20-30 °C.

In a preferred embodiment, the salt preparation step (ix) is carried out at 20-30 °C and wherein the reaction mass is stirred for 14-16 hour.

In a preferred embodiment, the process steps (I) to (ix) are performed in an inert atmosphere.

In some embodiments, the calcium salt of compound of formula (I) is crystalline. In preferred embodiments, the crystalline calcium salt of compound of formula (I) is an LLL-isomer.

In one embodiment, the crystalline calcium salt of compound of formula (I) is crystalline Form A. In a preferred embodiment, the crystalline Form A is characterized by an XRPD pattern comprising peaks with 2-theta values: 9.9±0.2, 13.2±0.2 and 19.8±0.2. Preferably, the crystalline Form A is further characterized by an XRPD pattern comprising peaks with 2-theta values: 9.9±0.2, 11.8±0.2, 12.9±0.2, 13.2±0.2, 19.8±0.2 and 23.9±0.2; or 9.9±0.2, 11.8±0.2, 13.2±0.2, 13.8±0.2, 19.2±0.2, 19.8±0.2 and 23.9±0.2. More preferably, the crystalline Form A is further characterized by an XRPD pattern comprising peaks with 2-theta values: 8.1±0.2, 9.9±0.2, 11.8±0.2, 12.9±0.2, 13.2±0.2, 13.8±0.2, 16.3±0.2, 16.5±0.2, 19.2±0.2, 19.8±0.2, 20.1±0.2 and 23.9±0.2; or 8.1±0.2, 9.9±0.2, 10.6±0.2, 11.8±0.2, 12.9±0.2, 13.2±0.2, 13.8±0.2, 16.3±0.2, 16.5±0.2, 17.7±0.2, 19.2±0.2, 19.8±0.2, 20.1±0.2, 21.5±0.2 and 23.9±0.2. In yet preferable embodiment, the crystalline Form A is characterized by an XRPD pattern comprising peaks with 2-theta values: 6.5±0.2, 6.8±0.2, 8.1±0.2, 9.5±0.2, 9.9=0.2, 10.6±0.2, 11.8±0.2, 12.9±0.2, 13.2±0.2, 13.8±0.2, 14.1±0.2, 15.1±0.2, 16.3±0.2, 16.5±0.2, 17.7±0.2, 18.0±0.2, 18.7±0.2, 19.2±0.2, 19.5±0.2, 19.8±0.2, 20.1±0.2, 20.5±0.2, 21.5±0.2, 22.1±0.2, 22.7±0.2, 23.5±0.2, 23.9±0.2, 24.4±0.2, 24.9±0.2, 25.7±0.2, 26.7±0.2, 27.2±0.2, 27.8±0.2, 28.5±0.2, 29.1±0.2, 29.9±0.2, 31.2±0.2, 32.2±0.2, 33.4±0.2, 35.1±0.2, 37.6±0.2 and 38.4=0.2.

In one embodiment, the crystalline calcium salt of compound of formula (I) is crystalline Form B. In a preferred embodiment, the crystalline Form B is characterized by an XRPD pattern comprising at least 3 or at least 5 peaks with 2-theta values selected from: 18.5±0.2, 18.9±0.2, 22.5=0.2, 23.5±0.2, 25.4±0.2, 27.9±0.2, 28.9±0.2, 31.7±0.2, 33.9±0.2, 37.7±0.2 and 42.4±0.2. More preferably, the crystalline Form B is characterized by an XRPD pattern comprising peaks with 2-theta values: 22.5=0.2, 23.5±0.2, 25.4±0.2, 31.7±0.2, 33.9±0.2 and 37.7±0.2; or 18.9±0.2, 22.5±0.2, 23.5±0.2, 25.4±0.2, 27.9±0.2, 28.9±0.2, 31.7±0.2, 33.9±0.2, 37.7±0.2 and 42.4±0.2; or 18.5±0.2, 18.9±0.2, 22.5±0.2, 23.5±0.2, 25.4±0.2, 27.9=0.2, 28.9±0.2, 31.7±0.2, 33.9±0.2, 37.7±0.2 and 42.4=0.2.

In one embodiment, the crystalline calcium salt of compound of formula (I) is crystalline Form C. In a preferred embodiment, the crystalline Form C is characterized by a XRPD pattern comprising peaks with 2-theta values: 42.3±0.2, 42.9±0.2, 43.2±0.2 and 43.8±0.2.

In some embodiments, the present disclosure provides a pharmaceutical composition comprising a calcium salt of compound of formula (I), in accordance with the disclosure and one or more pharmaceutically acceptable excipients.

In some embodiments, upon storage of the composition at 2-8 °C, the impurity A-1 remains less than about 3%, preferably less than about 1.3% as measured by HPLC (% area) for a period of 12 months, preferably 18 months.

In some embodiments, upon storage of the composition at 2-8 °C, the impurity A-1 remains less than about 3%, preferably less than about 1.3% as measured by HPLC (% area) for a period of 12 months, preferably 18 months, more preferably 24 months.

In some embodiments, upon storage of the composition at 25±2 °C/60±5% RH, the impurity A-1 remains less than about 3%, preferably less than about 1.3% as measured by HPLC (% area) for a period of 12 months, preferably 18 months.

In some embodiments, upon storage of the composition at 25±2 °C/60±5% RH, the impurity A-1 remains less than about 3%, preferably less than about 1.3% as measured by HPLC (% area) for a period of 12 months, preferably 18 months, more preferably 24 months.

In some embodiments, upon storage of the composition at 2-8 °C, the impurity A-2 remains less than about 1%, preferably less than about 0.60% as measured by HPLC (% area) for a period of 12 months, preferably 18 months.

In some embodiments, upon storage of the composition at 2-8 °C, the impurity A-2 remains less than about 1%, preferably less than about 0.60% as measured by HPLC (% area) for a period of 12 months, preferably 18 months, more preferably 24 months.

In some embodiments, upon storage of the composition at 25±2 °C/60%±5% RH, the impurity A-2 remains less than about 1%, preferably less than about 0.60% as measured by HPLC (% area) for a period of 12 months, preferably 18 months.

In some embodiments, upon storage of the composition at 25±2 °C/60%±5% RH, the impurity A-2 remains less than about 1%, preferably less than about 0.60% as measured by HPLC (% area) for a period of 12 months, preferably 18 months, more preferably 24 months.

In some embodiments, upon storage of the composition at 2-8 °C, the impurity A-3 remains less than about 1%, preferably less than about 0.60% as measured by HPLC (% area) for a period of 12 months, preferably 18 months.

In some embodiments, upon storage of the composition at 2-8 °C, the impurity A-3 remains less than about 1%, preferably less than about 0.60% as measured by HPLC (% area) for a period of 12 months, preferably 18 months, more preferably 24 months.

In some embodiments, upon storage of the composition at 25±2 °C/60%±5% RH, the impurity A-3 remains less than about 1%, preferably less than about 0.60% as measured by HPLC (% area) for a period of 12 months, preferably 18 months.

In some embodiments, upon storage of the composition at 25±2 °C/60%±5% RH, the impurity A-3 remains less than about 1%, preferably less than about 0.60% as measured by HPLC (% area) for a period of 12 months, preferably 18 months, more preferably 24 months.

In preferred embodiments, the calcium salt of compound of formula (I) according to one or more of above embodiments, wherein the compound of formula (I) is an LLL-isomer.

In some embodiments, the pharmaceutical composition in accordance with the disclosure is solid oral dosage form, preferably selected from tablet, capsule, oral liquid dosage form such as oral liquid solution or oral liquid suspension, powder or powder for oral suspension.

In some embodiments, the pharmaceutical composition in accordance with the disclosure is tablet.

In some embodiments, the present disclosure provides a tablet composition comprising a calcium salt of compound of formula (I) in accordance with the disclosure.

In some embodiments, upon storage of the tablet at 2-8 °C, the impurity A-1 remains less than about 3%, preferably less than about 1.3% as measured by HPLC (% area) for a period of 12 months, preferably 18 months.

In some embodiments, upon storage of the tablet at 2-8 °C, the impurity A-1 remains less than about 3%, preferably less than about 1.3% as measured by HPLC (% area) for a period of 12 months, preferably 18 months, more preferably 24 months.

In some embodiments, upon storage of the tablet at 25±2 °C/60±5% RH, the impurity A-1 remains less than about 3%, preferably less than about 1.3% as measured by HPLC (% area) for a period of 12 months, preferably 18 months.

In some embodiments, upon storage of the tablet at 25±2 °C/60±5% RH, the impurity A-1 remains less than about 3%, preferably less than about 1.3% as measured by HPLC (% area) for a period of 12 months, preferably 18 months, more preferably 24 months.

In some embodiments, upon storage of the tablet at 2-8 °C, the impurity A-2 remains less than about 1%, preferably less than about 0.60% as measured by HPLC (% area) for a period of 12 months, preferably 18 months.

In some embodiments, upon storage of the tablet at 2-8 °C, the impurity A-2 remains less than about 1%, preferably less than about 0.60% as measured by HPLC (% area) for a period of 12 months, preferably 18 months, more preferably 24 months.

In some embodiments, upon storage of the tablet at 25±2 °C/60%±5% RH, the impurity A-2 remains less than about 1%, preferably less than about 0.60% as measured by HPLC (% area) for a period of 12 months, preferably 18 months.

In some embodiments, upon storage of the tablet at 25±2 °C/60%±5% RH, the impurity A-2 remains less than about 1%, preferably less than about 0.60% as measured by HPLC (% area) for a period of 12 months, preferably 18 months, more preferably 24 months.

In some embodiments, upon storage of the tablet at 2-8 °C, the impurity A-3 remains less than about 1%, preferably less than about 0.60% as measured by HPLC (% area) for a period of 12 months, preferably 18 months.

In some embodiments, upon storage of the tablet at 2-8 °C, the impurity A-3 remains less than about 1%, preferably less than about 0.60% as measured by HPLC (% area) for a period of 12 months, preferably 18 months, more preferably 24 months.

In some embodiments, upon storage of the tablet at 25_2 °C/60%±5% RH, the impurity A-3 remains less than about 1%, preferably less than about 0.60% as measured by HPLC (% area) for a period of 12 months, preferably 18 months.

In some embodiments, upon storage of the tablet at 25±2 °C/60%±5% RH, the impurity A-3 remains less than about 1%, preferably less than about 0.60% as measured by HPLC (% area) for a period of 12 months, preferably 18 months, more preferably 24 months.

In preferred embodiments, the calcium salt of compound of formula (I) according to one or more of above embodiments, wherein the compound of formula (I) is an LLL-isomer.

The purity and/or impurities in the calcium salt of the compound of formula (I) is measured by HPLC (% area), preferably using Method A, characterized by:
Mobile phase-A: potassium dihydrogen phosphate in water having pH 6.5±0.05;
Mobile phase-B: 100% Acetonitrile;
Column: ZIC HILIC (250×4.6) mm, 5.0 μm 200 A°; and
preferably gradient elution at column temperature of 25 °C, flow rate of 1.0 mL/minute, injection volume of 10 μL, run time of 40 minutes, and gradient program as following:

| Time | Mobile | Mobile ) |
|---|---|---|
| (Minutes) | Phase-A (%) | Phase-B (% |
| 0 | 20 | 80 |
| 2 | 20 | 80 |
| 20 | 35 | 65 |
| 30 | 50 | 50 |
| 35 | 50 | 50 |
| 35.1 | 20 | 80 |
| 40 | 20 | 80 |

The chiral purity and/or impurities in the calcium salt of the compound of formula (I) is measured by chiral HPLC (% area), preferably using Method C, characterized by: Mobile phase: buffer of perchloric acid in water: methanol (90:10) v/v;
Column: Crown Pak CR-(+) (150*4.0 mm), 5 μm; and
preferably isocratic elution at column temperature of 15±2 °C, flow rate of 0.5 mL/min, injection volume of 10 μL.

In some embodiments, the present disclosure provides a calcium salt of compound of formula (I) or the composition according to the disclosure for use in treatment of cancer.

In some embodiments, the present disclosure provides a calcium salt of compound of formula (I) according to any of the preceding embodiments or the composition thereof for use in treatment of cancer.

In some embodiments, the present disclosure provides a method of treating cancer comprising administering a therapeutically effective amount of the calcium salt of compound of formula (I) according to the disclosure, or the composition according to the disclosure to a patient in need thereof.

In some embodiments, the present disclosure provides a method of treating cancer comprising administering a therapeutically effective amount of the calcium salt of compound of formula (I) or the composition according to any of the preceding embodiments to a patient in need thereof.

In some embodiments, the cancer is selected from breast cancer, lung cancer, colorectal cancer, prostate cancer, ovarian cancer, oropharyngeal cancer, nasopharyngeal cancer, cervical cancer, neuroendocrine cancer, gastrointestinal cancer, stomach cancer, pancreatic cancer and liver cancer.

In one embodiment, the cancer is a hematological cancer.

In one embodiment, the cancer is leukemia.

In one embodiment, the cancer is Acute Myeloid Leukemia (AML) or Myelodysplastic syndrome (MDS).

In yet another embodiment, AML is a newly diagnosed AML or a relapsed and/or refractory AML.

In some embodiments, the present disclosure provides a method of treating cancer associated fibrosis comprising administering a therapeutically effective amount of the calcium salt of compound of formula (I), or the composition according to any of the preceding embodiments to a patient in need thereof.

In some embodiments, the cancer-associated fibrosis is selected from biliary cancer, cholangiocarcinoma, bladder cancer, colorectal cancer, colon cancer, rectal cancer, colorectal adenocarcinoma, brain cancer, meningioma, glioma, astrocytoma, oligodendroglioma, medulloblastoma, breast cancer, adenocarcinoma of the breast, inflammatory carcinoma of the breast, papillary carcinoma of the breast, medullary carcinoma of the breast, kidney cancer nephroblastoma/Wilms' tumor, renal cell carcinoma, cervical cancer, squamous cell carcinoma of the cervix, cervical adenocarcinoma, gastric cancer, esophageal cancer, gastrointestinal stromal tumor (GIST), head and neck cancer, head and neck squamous cell carcinoma, oral cancer, oral squamous cell carcinoma (OSCC), keloids, leukemia, acute lymphocytic leukemia (ALL), acute myelogenous leukemia (AML), chronic myelogenous leukemia (CML), chronic lymphocytic leukemia (CLL), lung cancer bronchogenic carcinoma, small cell lung cancer (SCLC), non-small cell lung cancer (NSCLC), adenocarcinoma of the lung, liver cancer, hepatocellular cancer (HCC), malignant hepatoma, lymphoma, Hodgkin lymphoma (HL), non-Hodgkin lymphoma (NHL), follicular lymphoma, diffuse large B-cell lymphoma (DLBCL), mantle cell lymphoma (MCL), medulloblastoma, multiple myeloma (MM), myelodysplasia syndrome (MDS), myeloproliferative disorder (MPD), polycythemia vera (PV), essential thrombocythemia (ET), agnogenic myeloid metaplasia (AMM), primary myelofibrosis (PMF), chronic neutrophilic leukemia (CNL), hypereosinophilic syndrome (HES), neuroblastoma, neurofibroma, neurofibromatosis (NF) type 1, neurofibromatosis (NF) type 2, schwannomatosis, skin cancer, squamous cell carcinoma (SCC), keratoacanthoma (KA), melanoma, basal cell carcinoma (BCC), dermatofibroma, osteosarcoma, ovarian cancer, cystadenocarcinoma, ovarian embryonal carcinoma, ovarian adenocarcinoma, neuroendocrine cancer, gastroenteropancreatic neuroendocrine tumor (GEP-NET), carcinoid tumor, pancreatic cancer, pancreatic adenocarcinoma, intraductal papillary mucinous neoplasm (IPMN), prostate cancer, prostate adenocarcinoma, soft tissue tumors, angiolipoma, angioleiomyoma, malignant fibrous histiocytoma (MFH), liposarcoma, malignant peripheral nerve sheath tumor (MPNST), chondrosarcoma, fibrosarcoma, myosarcoma, osteosarcoma, and tumors associated with desmoplasia.

In some embodiments, the fibrosis is characterized by aberrant fibroblast proliferation and extracellular matrix deposition in the tissue of a subject.

In some embodiments, the fibrosis is further characterized by an inflammation in the tissue.

In some embodiments, the inflammation is acute or chronic inflammation.

In some embodiments, the present disclosure provides the oral administration of the calcium salt of compound of formula (I) or the composition according to any of the preceding embodiments at a total daily dose not exceeding 800 mg, preferably not exceeding 600 mg, more preferably not exceeding 500 mg, most preferably not exceeding 400 mg to a patient in need thereof for treating cancer.

In some embodiments, the present disclosure provides the oral administration of the calcium salt of compound of formula (I) or the composition according to any of the preceding embodiments at a total daily dose of 50 mg to 800 mg. In another embodiment, the method comprises the oral administration of the calcium salt of compound of formula (I) or the composition according to any of the preceding embodiments at an amount of 50 mg to 100 mg. In another embodiment, the total daily dose of the calcium salt of compound of formula (I) or the composition according to any of the preceding embodiments is 50 mg to 200 mg. In another embodiment, the total daily dose of the calcium salt of compound of formula (I) or the composition according to any of the preceding embodiments is 50 mg to 400 mg. In another embodiment, the total daily dose of the calcium salt of compound of formula (I) or the composition according to any of the preceding embodiments is 50 mg to 800 mg. In another embodiment, the total daily dose of the calcium salt of compound of formula (I) or the composition according to any of the preceding embodiments is 100 mg to 200 mg. In another embodiment, the total daily dose of the calcium salt of compound of formula (I) or the composition according to any of the preceding embodiments is 100 mg to 400 mg. In another embodiment, the total daily dose of the calcium salt of compound of formula (I) or the composition according to any of the preceding embodiments is 100 mg to 800 mg. In another embodiment, the total daily dose of the calcium salt of compound of formula (I) or the composition according to any of the preceding embodiments is 200 mg to 400 mg. In another embodiment, the total daily dose of calcium salt of compound of formula (I) or the composition according to any of the preceding embodiments is 200 mg to 800 mg. In another embodiment, the total daily dose of the calcium salt of compound of formula (I) or the composition according to any of the preceding embodiments is 400 mg to 800 mg. In another embodiment, the total daily dose of the calcium salt of compound of formula (I) or the composition according to any of the preceding embodiments is 300 mg to 600 mg. In a preferred embodiment, the total daily dose of the calcium salt of compound of formula (I) or the composition according to any of the preceding embodiments is 600 mg. In another embodiment, the method comprises the oral administration of the calcium salt of compound of formula (I) or the composition according to any of the preceding embodiments at a total daily dose of 50 mg, 60 mg, 70 mg, 80 mg, 90 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg or 800 mg.

In some embodiments, the present disclosure provides a use of the calcium salt of the compound of formula (I) according to the disclosure, or the composition according to the disclosure in the manufacture of a medicament for treatment of cancer.

In some embodiments, the present disclosure provides a use of the calcium salt of the compound of formula (I) according to any of the preceding embodiments, or the composition according to any of the preceding embodiments in the manufacture of a medicament for treatment of cancer.

In preferred embodiments, the calcium salt of compound of formula (I) according to one or more of above embodiments, wherein the compound of formula (I) is an LLL-isomer.

In some embodiments, the calcium salt of compound of formula (I) as disclosed in any of the preceding embodiments, wherein the compound of formula (I) is enantiomerically pure or chiral pure, preferably the compound of formula (I) is LLL-isomer.

### Pharmaceutical Compositions

In some embodiments, the disclosure relates to a pharmaceutical composition comprising the calcium salt of compound of formula (I) in accordance with the disclosure and one or more pharmaceutically acceptable excipients. In preferred embodiments, the calcium salt of compound of formula (I) is an LLL-isomer.

In some embodiments, the present disclosure relates to a pharmaceutical composition comprising the calcium salt of compound of formula (I) in accordance with the disclosure in combination with at least one other therapeutic agent and a pharmaceutically acceptable carrier or excipient.

In some embodiments, the present disclosure provides a use of pharmaceutical composition comprising the calcium salt of compound of formula (I) as described herein and at least one pharmaceutically acceptable excipient (such as a pharmaceutically acceptable carrier or diluent). The calcium salt of compound of formula (I), described in the present disclosure may be associated with a pharmaceutically acceptable excipient (such as a carrier or a diluent) or be diluted by a carrier or enclosed within a carrier which can be in the form of a capsule, sachet, paper or other container.

The compound described in the present disclosure are typically administered in the form of a pharmaceutical composition. Such compositions can be prepared using procedures well known in the pharmaceutical art and comprise at least one compound of the present disclosure. The pharmaceutical composition of the present disclosure comprises one or more compounds described herein and one or more pharmaceutically acceptable excipients. Typically, the pharmaceutically acceptable excipients are approved by regulatory authorities or are generally regarded as safe for human or animal use. The pharmaceutically acceptable excipients include, but are not limited to, carriers, diluents, glidants and lubricants, preservatives, buffering agents, chelating agents, polymers, gelling agents, viscosifying agents, solvents and the like.

The pharmaceutical composition can be administered by oral, parenteral or inhalation routes. Examples of the parenteral administration include administration by injection, percutaneous, transmucosal, trans nasal and transpulmonary administrations.

Examples of suitable carriers include, but are not limited to, water, salt solutions, alcohols, polyethylene glycols, lactose, sucrose, dextrin, magnesium carbonate, sugar, amylose, magnesium stearate, talc, stearic acid, lower alkyl ethers of cellulose, silicic acid, fatty acids, fatty acid amines, fatty acid monoglycerides and diglycerides, fatty acid esters and polyoxyethylene.

The pharmaceutical composition may also include one or more pharmaceutically acceptable auxiliary agents, wetting agents, suspending agents, preserving agents, buffers, sweetening agents, flavouring agents, colorants or any combination of the foregoing. The pharmaceutical compositions may be in conventional forms, for example, tablets, capsules, solutions, suspensions, injectables or products for topical application. Further, the pharmaceutical composition of the present disclosure may be formulated to provide desired release profile.

Administration of compounds of the disclosure, in pure form or in an appropriate pharmaceutical composition, can be carried out using any of the accepted routes of administration. The route of administration may be any route which effectively transports the compound of the present disclosure to the appropriate or desired site of action. Suitable routes of administration include, but are not limited to, oral, nasal, buccal, dermal, intradermal, transdermal, parenteral, rectal, subcutaneous, intravenous, intraurethral, intramuscular or topical.

Solid oral formulations include, but are not limited to, tablets, capsules (soft or hard gelatin), dragees (containing the active ingredient in powder or pellet form), troches and lozenges.

Liquid formulations include, but are not limited to, syrups, emulsions and sterile injectable liquids, such as suspensions or solutions.

Topical dosage forms of the compounds include ointments, pastes, creams, lotions, powders, solutions, eye or ear drops, impregnated dressings and may contain appropriate conventional additives such as preservatives, solvents to assist drug penetration.

The pharmaceutical compositions of the present disclosure may be prepared by conventional techniques known in literature.

### ABBREVIATIONS

The abbreviations used in the entire specification is summarized below with their meaning.
XRPD—X-Ray Powder Diffraction; HPLC—High-performance liquid chromatography; ICP-MS—Inductively coupled plasma mass spectrometry; NMR—Nuclear Magnetic Resonance Spectroscopy; LC-MS—Liquid chromatography-mass spectrometry; RRT—Relative Retention Time; NMT—Not more than; NLT—Not less than; ND—Not detected; BDL—Below detection limit; BQL—Below quantitation limit; ICH—International Council for Harmonization of Technical Requirements for Pharmaceuticals for Human Use; TLC—Thin Layer Chromatography; ECF—Ethylchloroformate; NMM—N-methylmorpholine; DCM—Dichloromethane; TFA—Trifluroacetic anhydride; DIPEA—N,N-Di-isopropylethylamine; DIC—N,N-Di isopropyl carbodiimide; DMF—N,N-Dimethyl formamide; Fmoc—Fluorenylmethoxycarbonyl; Pbf—2,2,4,6,7-pentamethyldihydrobenzofuran-5-sulfonyl; MTBE—methyl tert-butyl ether; CDI—N,N′-Carbonyl diimidazole; DMAP—4-dimethylamino pyridine; TIPS—Triisopropyl silane.

### EXPERIMENTAL

The present invention provides methods for the preparation of the calcium salt of the compound of formula (I) according to the procedures of the following examples, using appropriate materials and conditions. Those skilled in the art will understand that known variations of the conditions and processes of the following preparative procedures can be used to prepare these compounds of the present invention. Moreover, by utilizing the procedures described in detail, one of ordinary skill in the art can prepare additional compounds of the present invention.

### Example 1: Preparation of Compound of Formula (I) Calcium Salt

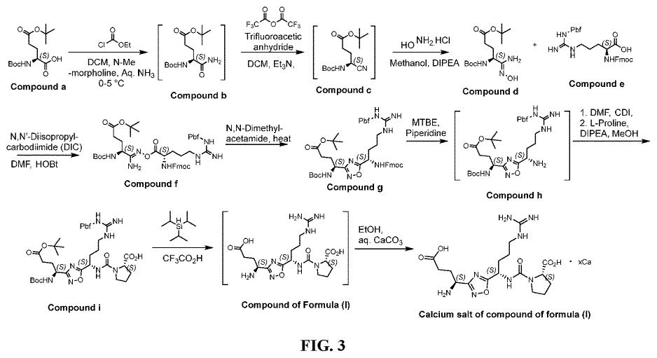

FIG. 3 discloses a scheme of preparation of the compound of formula (I) Calcium Salt. As disclosed in the FIG. 3, following are the steps involved:

### Preparation of tert-butyl(S)-5-amino-4-((tert-butoxycarbonyl)amino)-5-oxopentanoate (Compound b)

Ethylchloroformate (ECF) (1.2 eq.) and N-methylmorpholine (NMM) (1.2 eq.) were added to a solution of(S)-5-(tert-butoxy)-2-((tert-butoxycarbonyl)amino)-5-oxopentanoic acid (compound a) (1.0 eq.) in Dichloromethane (DCM) (6 vol.) and stirred at −25 °C to −15 °C for 30-40 min. 2 vol. of aq. ammonia was then added to the active reaction mass resulting from the reaction and the reaction mass was stirred at −5 °C to 5 °C for 60 min to 90 min. The completion of the reaction was confirmed by TLC analysis. After completion of the reaction citric acid solution is added to the reaction mass and then the temperature of the reaction mass was raised and stirred for 20 min to 30 min. The reaction mixture was then allowed to settle and lower DCM layer and top aqueous layer were collected separately. The organic layer was washed with aq. sodium bicarbonate solution and water. The separated organic layer was dried over sodium sulphate to obtain compound b.

### Preparation of tert-butyl(S)-4-((tert-butoxycarbonyl)amino)-4-cyanobutanoate (Compound c)

Trifluroacetic anhydride (TFA) (3 eq.) was added to a solution of compound b (1 eq.), triethylamine (3 eq.) in DCM (6 vol.) at −5 to 5 °C. The reaction mass was stirred for 2 to 3 hours. The completion of the reaction was confirmed by TLC analysis. After completion of the reaction purified water was added (5 vol.) into the reaction mass and then the reaction mass temperature was raised to 20 to 30 °C. And after 30 min the organic layer was separated and was washed with aq. sodium bicarbonate solution followed by citric acid solution and water. The separated organic layer was distilled to obtain compound c, which was used for next step directly.

### Preparation of tert-butyl (S,Z)-5-amino-4-((tert-butoxycarbonyl)amino)-5-(hydroxyimino) pentanoate (Compound d)

Hydroxylamine hydrochloride (2 eq.), and N, N-Di-isopropyl ethylamine (DIPEA) (3.0 eq.) were added to a solution of compound c (1 eq.) in methanol (3 vol.) and stirred at 20 to 30 °C for 4 to 5 hours. The completion of the reaction was confirmed by TLC analysis. After the completion of reaction, the compound from the water was extracted by using ethyl acetate (10 vol.) followed washing the organic layer with brine solution. The organic layer was swapped thrice with hexane (3 vol). The product was isolated with ethyl acetate and hexane (1:20) and the isolated solid was dried to obtain compound d as product.

### Preparation of tert-butyl (5S,10S,Z)-9-amino-10-((tert-butoxycarbonyl)amino)-1-(9H-fluoren-9-yl)-3,6-dioxo-5-(3-(3-((2,2,4,6,7-pentamethyl-2,3-dihydrobenzofuran-5-yl) sulfonyl)guanidino)propyl)-2,7-dioxa-4,8-diazatridec-8-en-13-oate (Compound f)

1-Hydroxybenzotriazole monohydrate (HOBt·H₂O) (1.05 eq.) and N,N-Di isopropyl carbodiimide (DIC) (1.05 eq.) were added to a solution of N²-(((9H-fluoren-9-yl)methoxy)carbonyl)-N^{w}-((2,2,4,6,7-pentamethyl-2,3-dihydrobenzofuran-5-yl)sulfonyl)-L-arginine or Fmoc-Arg(pbf)-OH (compound e) (0.7 eq.) in DMF (6 vol.) at 0 to 10 °C and stirred for 20 to 30 min. Compound d (1.0 eq.) was added to the reaction mass at the same temperature followed by raising the temperature to 20 to 30 °C and stirring for 4 to 5 hours. The completion of the reaction was confirmed by TLC analysis. The reaction mixture was quenched with purified water and stirred for 8-10h. The residue was washed filtered and dried under vacuum to obtain compound f.

### Preparation of tert-butyl(S)-4-(5-((S)-1-((((9H-fluoren-9-yl)methoxy)carbonyl)amino)-4-(3-((2,2,4,6,7-pentamethyl-2,3-dihydrobenzofuran-5-yl)sulfonyl)guanidino)butyl)-1,2,4-oxadiazol-3-yl)-4-((tert-butoxycarbonyl)amino)butanoate (Compound g)

N,N-Dimethyl formamide (DMF) (15 vol.) was added to a solution of compound f (51 eq.) in purified water (7.5 vol.) at room temperature and the reaction mass was heated to 78±2 °C for 5.5 hours to 6.5 hours. The completion of the reaction was confirmed by TLC analysis. After completion of reaction, the reaction mass was reverse quenched to purified water at 5±5 °C. The reaction mass was then stirred for 16 to 18 hours. The solid product was then precipitated, washed with purified water. The compound was then dried in Vacuum Tray Dryer to obtain compound g.

### Preparation of tert-butyl(S)-4-(5-((S)-1-amino-4-(3-((2,2,4,6,7-pentamethyl-2,3-dihydrobenzofuran-5-yl)sulfonyl)guanidino)butyl)-1,2,4-oxadiazol-3-yl)-4-((tert-butoxycarbonyl)amino)butanoate (Compound h)

Compound g (1 eq.) was added to piperidine (2.0 vol.) and methyl tert-butyl ether (MTBE) (10 vol.) at 20-30 °C and the reaction mass was stirred at 5±5 °C for 2 to 3 hours. The completion of the reaction was confirmed by TLC analysis. After completion, the reaction mixture was treated with 10% citric acid solution followed by bicarbonate solution wash, brine wash and treatment with SC-40 carbon (10 w %). The organic mass was separated, dried over sodium sulphate (20 w %) and distilled to obtain the crude product of compound h.

### Preparation of (((S)-1-(3-((S)-4-(tert-butoxy)-1-((tert-butoxycarbonyl)amino)-4-oxobutyl)-1,2,4-oxadiazol-5-yl)-4-(3-((2,2,4,6,7-pentamethyl-2,3-dihydrobenzofuran-5-yl)sulfonyl)guanidino)butyl)carbamoyl)-L-proline (Compound i)

N,N′-Carbonyl diimidazole (CDI) was added to a solution of compound h in DMF (2 vol.) and the resulting solution was stirred at room temperature for 7 to 8 hours. The reaction mixture was cooled to 5±5 °C and L-proline solution (L-proline, N,N-Diisopropylethylamine in methanol) (10.0 vol) was added to it. 4-dimethylamino pyridine (DMAP) was added to the above reaction mixture 5±5 °C and stirred for 4-5 hours. After completion of reaction (confirmed by TLC), it was diluted with ethyl acetate (20.0 L) and brine at 5±5 °C, stirred for 20-30 min. at 20 to 30 °C and the layer were separated. The organic layer was dried over sodium sulphate. The mass was added to Heptane and stirred for 10-12 hours, the residue was filtered and dried under vacuum for 1-3 hours to obtain compound i.

### Deprotection of Compound i to afford (((S)-1-(3-((S)-1-amino-3-carboxypropyl)-1,2,4-oxadiazol-5-yl)-4-guanidinobutyl)carbamoyl)-L-proline (Compound of formula (I))

Compound i (1 eq.) was added to a cocktail mixture of Trifluoroacetic acid (TFA) (3.75 vol.), Triisopropyl silane (TIPS) (0.25 col.) and water (0.25 vol.) and was stirred at 5±5 °C followed by stirring at room temperature for 2-3 hours. The resulting reaction mixture was vacuum dried, diluted with IPA and filtered to yield the compound of formula (I).

### Preparation of the Calcium Salt of the Compound of Formula (I)

Calcium carbonate was added to an aqueous solution of Compound of formula (I) adjusted at 10±5 °C. The reaction mixture was stirred for 14 to 16 hours at 20-30 °C and filtered. The filtrate containing compound of formula (I) Calcium was precipitated from the solution using ethanol as an antisolvent and filtered at room temperature. The solid, so obtained, was dried at 20-30 °C under vacuum (NLT 600 mm/Hg) for 18-20 hours under argon atmosphere. The dried material was sieved to obtain the calcium salt of the compound of formula (I).

### Chromatographic Purity and Related Substances by HPLC:

| Test Name | Acceptance criteria |
|---|---|
| Purity and | i) Purity by HPLC: NLT 97.0 |
| Related | ii) Impurity at 0.25 RRT: NMT 0.50 |
| substances | iii) Impurity at 0.62 RRT: NMT 1.30 |
| | iv) Impurity at 0.72 RRT: NMT 0.60 |
| | v) Impurity at 0.86 RRT: NMT 0.50 |
| | vi) Any single unspecified impurity: NMT 0.50 |

### Method for Purity and Related Substances by HPLC (% Area):

Method A: For Calcium salt of compound of formula (I)
Mobile phase-A: Weigh and transfer about 1.36 g of Potassium dihydrogen phosphate in a 1000 mL of water, dissolve by sonication and adjust the pH 6.5=0.05 with potassium hydroxide solution then filter the mobile phase through 0.45 μm membrane and degas it.
Mobile phase-B: 100% Acetonitrile
Preparation of Diluent: 100% Milli-Q water

### Chromatographic Conditions:

| Column: | ZIC HILIC | |
|---|---|---|
| | (250X 4.6) | mm, |
| | 5.0 | μm |
| | 200 | Aº |
| Injection volume: | 10 | μL |
| Flow rate: | 1.0 | mL/minute |
| Column temperature: | 25 | °C |
| Detection Wavelength: | 210 | nm |
| Run Time | 40 | Minutes |

| | |
|---|---|
| Needle wash | Milli-Q water |

### Gradient Program:

| Time | Mobile | Mobile |
|---|---|---|
| (Minutes) | Phase-A (%) | Phase-B (%) |
| 0 | 20 | 80 |
| 2 | 20 | 80 |
| 20 | 35 | 65 |
| 30 | 50 | 50 |
| 35 | 50 | 50 |
| 35.1 | 20 | 80 |
| 40 | 20 | 80 |

Blank: use diluent as blank
Note: Increase the flow rate gradually to 1.0 mL/min in about 5 min with incremental flow rate of 0.2 mL/min

### Preparation of Calcium Salt of Compound of Formula (I) Standard Solution:

Weigh accurately 75 mg of calcium salt of compound of formula (I) standard and transfer into 25 mL volumetric flask, dissolve with 15 mL of diluent by sonication and dilute up to the mark with diluent. Mix well

### Preparation of Sample Solution (Duplicate Preparation):

Weigh accurately 75 mg of test sample and transfer into 25 mL volumetric flask, dissolve with 15 mL of diluent by sonication and dilute up to the mark with diluent. Mix well.

### Procedure:

Before initiating sequence wash the column with ACN: H₂O (50:50) (% v/v) for 1 h followed by ACN: H2O (80:20) (% v/v) for about 2 h, then equilibrate the chromatographic system with initial gradient of mobile phase (i.e; MP-A: MP-B (20:80)) at least for 2 h till stable baseline is observed. Inject blank, standard and test sample solutions as per the below sequence.

Method B: For Pharmaceutical composition comprising Calcium salt of compound of formula (I) Mobile phase-A: Weigh and transfer about 1.36 g of Potassium dihydrogen phosphate in a 1000 mL of water, mix well and sonicate for 5 minutes then filter the mobile phase through 0.45 μm membrane filter.

Mobile phase-B: 100% Acetonitrile

Preparation of Diluent: Mix 500 mL of water and 500 mL of Acetonitrile into the 1000 mL mobile phase bottle and sonicate for 5 minutes.

### Chromatographic Conditions:

| Column: | ZIC HILIC | |
|---|---|---|
| | (250X4.6) | mm, |
| | 5 | μm |
| Injection volume: | 20 | μL |
| Flow rate: | 1.0 | mL/minute |
| Column temperature: | 45 °C ± 2 °C | |
| Sample cooler temperature: | 10 °C ± 2 °C | |
| Detection Wavelength: | 210 | nm |
| Run Time | 40 | Minutes |

| | |
|---|---|
| Needle wash solution | Acetonitrile:Water (40:60% v/v) |
| Seal wash solution | Acetonitrile:Water (50:50% v/v) |
| Needle wash time | Extended |

### Gradient Program:

| Time | Mobile | Mobile |
|---|---|---|
| (Minutes) | Phase-A (%) | Phase-B (%) |
| 0 | 20 | 80 |
| 2 | 20 | 80 |
| 20 | 40 | 60 |
| 22 | 50 | 50 |
| 32 | 50 | 50 |
| 32.1 | 20 | 80 |
| 40 | 20 | 80 |

Blank: use diluent as blank

### Chiral Analysis by HPLC:

| Test Name | Acceptance criteria |
|---|---|
| Chiral analysis | i) Calcium salt of compound of formula |
| | (I) Chiral Purity: NLT 98.0 |
| | ii) RRT about 0.86 (LDL Isomer): NMT 1.5 |
| | iii) Sum of all isomers (Except LDL): NMT 1.0 |

### Chiral HPLC

### Method C

Preparation of buffer solution: Pipette out 9.7 mL of 70% perchloric acid and transfer into mobile phase bottle containing 1000 mL of milli-q-water, mixed well by sonication. Transfer 316 mL of above solution into 1000 mL mobile phase bottle and add 684 mL of milli-q-water, mix well by sonication, and filter the mobile phase through 0.45u filter paper.

Column washing procedure: Before and after analysis column shall be conditioned with mobile phase for 60 min. Any other solvents shall not be used for column washing.

### Chromatographic Conditions:

| Column: | Crown Pak CR-(+) | |
|---|---|---|
| | (150*4.0 | mm), |
| | 5 | μm |
| | (Part no: 27714) | |
| Mobile phase | Buffer:Méthanol (90:10) (v/v) | |
| Elution mode | Isocratic | |
| Column temperature | 15 ± 2 °C | |
| Flow rate | 0.5 | mL/min |
| Wavelength (2) | 210 | nm |
| Injection volume | 10 | μL |
| Run time | 80 | minutes |

| | |
|---|---|
| Diluent | Milli-Q-water (100%) |
| Retention times | calcium salt of compound of formula (I) |
| | (LLL isomer) is about 46.9 min |
| | calcium salt of compound of formula (I) |
| | (DLD isomer) is about 17.5 min (RRT about 0.37) |
| | calcium salt of compound of formula (I) |
| | (DDL isomer) is about 18.5 min (RRT about 0.39) |
| | calcium salt of compound of formula (I) |
| | (DLL isomer) is about 21.9 min (RRT about 0.47) |
| | calcium salt of compound of formula (I) |
| | (DDD isomer) is about 22.9 min (RRT about 0.49) |
| | calcium salt of compound of formula (I) |
| | (LLD isomer) is about 34.3 min (RRT about 0.73) |
| | calcium salt of compound of formula (I) |
| | (LDL isomer) is about 39.3 min (RRT about 0.84) |
| | calcium salt of compound of formula (I) |
| | (LDD isomer) is about 58.8 min (RRT about 1.25) |

Note:
Before connecting the column, the entire HPLC system including the injector and injector loop must be flushed with ethanol followed by 100% distilled water each for 2 hours to ensure the solvent free lines.

### Preparation of System Suitability Stock Solution:

Weigh accurately each 7 mg of DDD isomer, DDL isomer, LLD isomer, LDL isomer, DLD isomer, LDD isomer and DLL isomer standards into 20 mL volumetric flask, dissolve with 10 mL of diluent by sonication and dilute up to the mark with diluent.

Preparation of System suitability solution: Weigh accurately 37.5 mg of Calcium salt of compound of formula (I) standard and transfer into 5 mL volumetric flask, dissolve with 2 mL of diluent by sonication and add 3 mL of system suitability stock solution then dilute up to the mark with diluent.

Preparation of Sample solution: Weigh accurately 75 mg of test sample and transfer into 10 mL volumetric flask, dissolve with 5 of diluent by sonication and dilute up to the mark with diluent. Mix well.

Procedure: Equilibrate the chromatographic system till stable base line is observed. Inject the solutions as per the below sequence

### Elemental Impurities Content by ICP-MS:

| | Acceptance |
|---|---|
| Test | Criteria |
| Elemental | |
| impurities | |
| content by | |
| ICP-MS (ppm) | |
| Vanadium (V) | Not more than 10 |
| Cobalt (Co) | Not more than 5 |
| Nickel (Ni) | Not more than 20 |
| Arsenic (As) | Not more than 1.5 |
| Cadmium (Cd) | Not more than 0.5 |
| Mercury (Hg) | Not more than 3 |
| Lead (Pb) | Not more than 0.5 |
| Palladium (Pd) | Not more than 10 |

Inductively coupled plasma mass spectrometry (ICP-MS) Method D:

### Objective:

Determination of elemental impurities content of calcium salt of compound of formula (I) by ICP-MS (ppm).

### Apparatus/Reagents/Chemicals/Materials

Inductively Coupled Plasma Mass Spectrometer (ICP-MS).
Make: Agilent Technologies & Model: 7800 or equivalent.
Semi Micro Balance/Analytical Balance.
Make: Shimadzu or equivalent.
Nitric acid (69%) Suprapur or equivalent.
Vanadium (V), Cobalt (Co), Nickel (Ni), Arsenic (As), Cadmium (Cd), Mercury (Hg), Lead (Pb), Palladium (Pd), Yttrium (Y), Gold (Au) and Bismuth (Bi) certified reference materials traceable to NIST or equivalent.
Milli-Q-water or Equivalent.
Micropipette/Pipettes (1.0 mL, 0.2 mL)
Tubes-Tarson or Equivalent.
Class A-Pipettes.
Polypropylene tubes (15 mL, 50 mL)/Volumetric flasks (Class-A) (PMP)
Gloves, Nose mask and Personal care.

### Reagents/Standards

Cadmium ICP Standard (NIST Traceable).
Lead ICP Standard (NIST Traceable).
Arsenic ICP Standard (NIST Traceable).
Mercury ICP Standard (NIST Traceable).
Cobalt ICP Standard (NIST Traceable).
Vanadium ICP Standard (NIST Traceable).
Nickel ICP Standard (NIST Traceable).
Palladium ICP Standard (NIST Traceable).
Gold ICP Standard (NIST Traceable).
Yttrium ICP Standard (NIST Traceable).
Bismuth ICP Standard (NIST Traceable).

### Chemicals

Nitric acid Concentrated (About 65%), Grade: Suprapur or Equivalent.
Milli-Q Water (In house) or Equivalent.
1-Propanol: HPLC grade or Equivalent

### Equipment

ICPMS, Made: Agilent or equivalent, Model: 7800 or Equivalent
Analytical balance, Made: Sartorius or Equivalent

Description of Analytical method (Methodology)

| | | |
|---|---|---|
| Mass of Cadmium: | 111 | amu |
| Mass of Lead: | 208 | amu |
| Mass of Arsenic: | 75 | amu |
| Mass of Mercury: | 201 | amu |
| Mass of Cobalt: | 59 | amu |
| Mass of Vanadium: | 51 | amu |
| Mass of Nickel: | 60 | amu |
| Mass of Palladium: | 105 | amu |
| Mass of Gold: | 197 | amu |
| Mass of Yttrium: | 89 | amu |
| Mass of Bismuth: | 209 | amu |

Acquisition Parameters

| Acq mode: | Spectrum | |
|---|---|---|
| Peak pattern: | 3 | points |
| No. of Replicates: | 3 | |
| Integration time (Sec): | 0.3000 | |
| Stabilization time: | 10 | seconds |
| RF Power: | 1550 | W |
| RF Matching: | 1.10 | V |
| Sample depth: | 8.0 | mm |
| Nebulizer gas (Ar): | 1.07 | L/min |
| Peri pump settings: | | |
| Pre-Run: | | |
| Uptake speed (Nebulizer pump): | 0.30 | rps |
| Uptake time: | 60 | seconds |
| Stabilize: | 40 | seconds |

Post Run (Probe rinse)

| | | |
|---|---|---|
| Rinse speed (Nebulizer Pump): | 0.30 | rps |
| Rinse at rinse port (sample): | 10 | seconds |
| Rinse at rinse port (standard): | 10 | seconds |

Post Run (Rinse):

| Rinse vial 1 (Diluent): | 1 | |
|---|---|---|
| Rinse speed (Nebulizer Pump): | 0.30 | rps |
| Rinse at rinse vial (step 1): | 60 | seconds |
| Rinse at rinse port (step 1): | 10 | seconds |
| Rinse vial 2 (Diluent): | 2 | |
| Rinse speed (Nebulizer Pump): | 0.30 | rps |
| Rinse at rinse vial (step 2): | 60 | seconds |
| Rinse at rinse port (step 2): | 10 | seconds |
| Intelligent Rinse: | Off | |
| Pre-emptive rinse: | Off | |
| He Flow rate: | 4.3 | mL/min |

TABLE NO 1
List of elements and Specifications

| Sr. | | | Limit | | Mass | ISTD |
|---|---|---|---|---|---|---|
| No. | Element Name | Type | (ppm) | | (m/z) | (m/z) |
| 1 | Vanadium (V) | Analyte | NMT | 10 | 51 | 89 |
| 2 | Cobalt (Co) | Analyte | NMT | 5 | 59 | 89 |
| 3 | Nickel (Ni) | Analyte | NMT | 20 | 60 | 89 |
| 4 | Arsenic (As) | Analyte | NMT | 1.5 | 75 | 89 |
| 5 | Cadmium (Cd) | Analyte | NMT | 0.5 | 111 | 89 |
| 6 | Palladium (Pd) | Analyte | NMT | 10 | 105 | 89 |
| 7 | Mercury (Hg) | Analyte | NMT | 3 | 201 | 209 |
| 8 | Lead (Pb) | Analyte | NMT | 0.5 | 208 | 209 |

| | | | | | |
|---|---|---|---|---|---|
| 9 | Yttrium (Y) | Internal | NA | 89 | NA |
| | | standard | | | |
| 10 | Bismuth (Bi) | Internal | NA | 209 | NA |
| | | Standard | | | |
| 11 | Gold (Au) | NA | NA | 197 | NA |

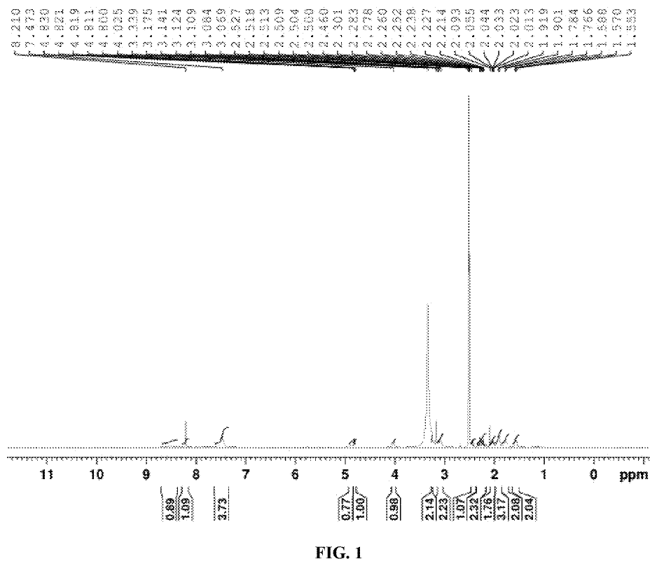

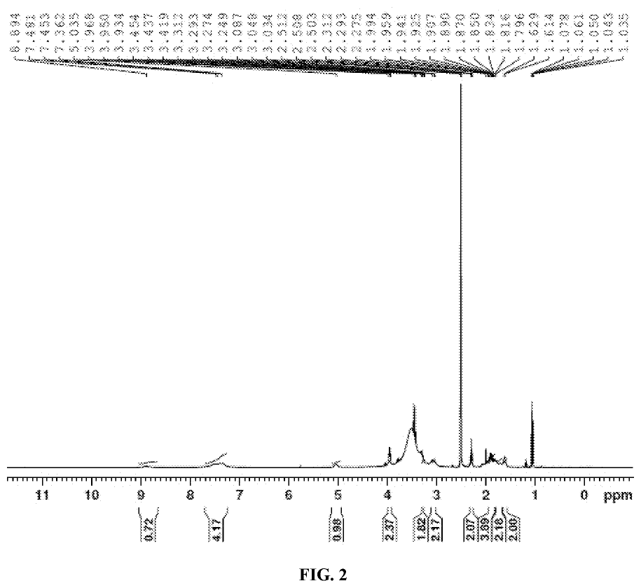

¹H NMR spectrum for the impurity A-1 is provided in FIG. 1, and ¹H NMR spectrum for the LDL isomer impurity is provided in FIG. 2.

The impurities A-1, A-2, A-3 and LDL isomer are confirmed by LC-MS as shown in table no. 2.

TABLE NO 2
List of specified impurities in the calcium salt of compound of formula (I)

| Impurity | | |
|---|---|---|
| Name | Structure | Mass (m/z) |
| A-1 | 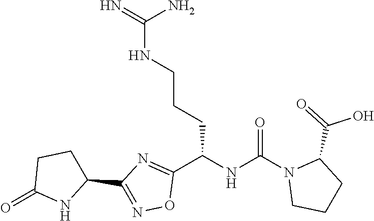 [SMILES: N=C(N)NCCC[C@H](NC(=O)N1CCC[C@H]1C(=O)O)c1nc([C@@H]2CCC(=O)N2)no1] | 423 |
| A-2 | 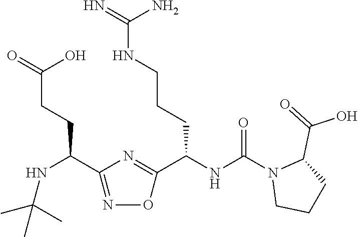 [SMILES: CC(C)(C)N[C@@H](CCC(=O)O)c1noc([C@H](CCCNC(=N)N)NC(=O)N2CCC[C@H]2C(=O)O)n1] | 497 |
| A-3 | 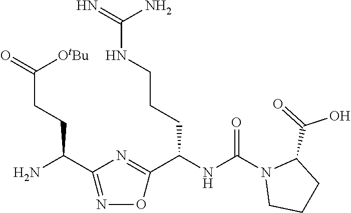 [SMILES: CC(C)(C)OC(=O)CC[C@H](N)c1noc([C@H](CCCNC(=N)N)NC(=O)N2CCC[C@H]2C(=O)O)n1] | 497 |
| LDL Isomer | 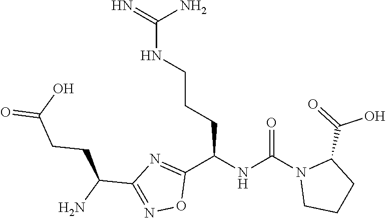 [SMILES: N=C(N)NCCC[C@@H](NC(=O)N1CCC[C@H]1C(=O)O)c1nc([C@@H](N)CCC(=O)O)no1] | 441 |

TABLE No. 3
Analytical Results for Impurities in Calcium Salt of Compound
of Formula (I) in five different batches

| | | Batch No. | | | | |
|---|---|---|---|---|---|---|
| | | 1 | 2 | 3 | 4 | 5 |
| | Acceptance | Results | Results | Results | Results | Results |
| Impurities | Criteria | (% area) | (% area) | (% area) | (% area) | (% area) |
| Purity of | NLT 97.0 | 98.76 | 98.72 | 99.68 | 99.3 | 99.6 |
| the Com- | | | | | | |
| pound | | | | | | |
| (I) Ca Salt | | | | | | |
| Impurities | NMT 0.50 | 0.07 | 0.08 | ND | 0.07 | BDL |
| at RRT | | | | | | |
| 0.25 | | | | | | |
| Impurities | NMT 1.30 | 0.16 | 0.39 | 0.14 | 0.09 | BQL |
| at RRT | | | | | | |
| 0.62 | | | | | | |
| Impurities | NMT 0.60 | 0.39 | 0.17 | 0.06 | ND | ND |
| at RRT | | | | | | |
| 0.72 | | | | | | |
| Impurities | NMT 0.50 | 0.38 | 0.24 | 0.08 | 0.13 | 0.10 |
| at RRT | | | | | | |
| 0.86 | | | | | | |
| Any Un- | NMT 0.50 | 0.13 | 0.13 | 0.04 | 0.10 | 0.07 |
| specified | | | | | | |
| impurity | | | | | | |

ND: Not detected,
BDL: Below detection limit,
BQL: Below quantitation limit.

Impurities observed from the various batches of the calcium salt of compound of formula (I) are summarized in table no: 3. Analytical results for impurities in batches of calcium salt of compound of formula (I) are from batch release analysis as presented in table no: 3. The data as disclosed in the table no: 3 shows that in case of multiple batches of the calcium salt of the compound of formula (I) the impurity formation is well below the acceptance criteria corresponding to each RRT and the purity of the calcium salt of the compound of formula (I) is in the range of 98.7% to 99.7% (HPLC % area).

### Stability Studies

The calcium salt of compound of formula (I) was manufactured and subjected to storage stability studies under long-term (2-8 °C) and accelerated conditions (23-27 °C/60%±5% (relative humidity [RH]) as per ICH conditions for up to 24 months.

The results are as follows, as shown in Table No: 4 and Table No: 5

TABLE No. 4
Long-term Storage Stability of the Calcium Salt of the Compound of Formula (I)
Storage at 2-8 °C

| | | | | | | | | | |
|---|---|---|---|---|---|---|---|---|---|
| Test | Acceptance | | 1^{st} | 3^{rd} | 6^{th} | 9^{th} | 12^{th} | 18^{th} | 24^{th} |
| Parameters | Limit | Initial | Month | Month | Month | Month | Month | Month | Month |

Purity and Related substances by HPLC (% area) by Method A:

| | | | | | | | | | |
|---|---|---|---|---|---|---|---|---|---|
| Purity | NLT 97.0 | 99.3 | 99.1 | 99.2 | 99.0 | 99.1 | 99.2 | 99.2 | 99.2 |
| Impurity at | NMT 0.50 | ND | ND | ND | ND | ND | ND | ND | ND |
| 0.25 RRT | | | | | | | | | |
| Impurity at | NMT 1.30 | 0.14 | 0.16 | 0.17 | 0.21 | 0.23 | 0.18 | 0.27 | 0.21 |
| 0.62 RRT | | | | | | | | | |
| Impurity at | NMT 0.60 | ≤0.05 | ND | 0.06 | ≤0.05 | 0.05 | ≤0.05 | 0.18 | ≤0.05 |
| 0.72 RRT | | | | | | | | | |
| Impurity at | NMT 0.50 | 0.07 | 0.09 | 0.06 | 0.07 | 0.11 | 0.10 | 0.19 | 0.15 |
| 0.86 RRT | | | | | | | | | |
| Any | NMT 0.50 | — | — | — | — | — | — | ≤0.05 | ≤0.05 |
| single un- | | | | | | | | | (RRT |
| specified | | | | | | | | | 0.37) |
| impurity | | — | — | — | — | — | — | ≤0.05 | ≤0.05 |
| | | | | | | | | | (RRT |
| | | | | | | | | | 0.42) |
| | | 0.09 | 0.17 | 0.12 | — | 0.06 | 0.17 | — | 0.16 |
| | | | | | | | | | (RRT |
| | | | | | | | | | 0.56) |
| | | — | — | — | — | 0.06 | — | ≤0.05 | — |
| | | | | | | (RRT | | (RRT | |
| | | | | | | 0.62) | | 0.68) | |
| | | 0.16 | 0.21 | 0.14 | 0.20 | 0.25 | 0.13 | — | 0.17 |
| | | | | | | | | | (RRT |
| | | | | | | | | | 0.80) |
| | | 0.07 | 0.12 | 0.07 | 0.09 | — | ≤0.05 | — | — |
| | | — | — | — | 0.06 | — | 0.06 | ≤0.05 | — |
| | | — | — | — | — | — | — | ≤0.05 | ≤0.05 |

Chiral analysis by HPLC (% area) by Method C:

| | | | | | | | | | |
|---|---|---|---|---|---|---|---|---|---|
| Chiral | NLT 98.0 | 99.2 | 99.2 | 99.3 | 99.3 | 99.2 | 99.3 | 99.2 | 99.2 |
| purity | | | | | | | | | |
| RRT | NMT 1.5 | 0.70 | 0.71 | 0.65 | 0.69 | 0.71 | 0.72 | 0.70 | 0.69 |
| about 0.86 | | | | | | | | | |
| (LDL | | | | | | | | | |
| Isomer) | | | | | | | | | |
| Sum of all | NMT 1.0 | 0.08 | 0.06 | 0.05 | 0.05 | 0.05 | ND | 0.09 | 0.09 |
| other | | | | | | | | | |
| isomers | | | | | | | | | |
| (except | | | | | | | | | |
| LDL | | | | | | | | | |
| isomer) | | | | | | | | | |

NMT: not more than;
NLT: not less than;
ND: not detected

TABLE No. 5
Storage Stability of the Calcium Salt of the Compound of Formula (I) at Accelerated Conditions
Storage at 23-27 °C and 60% ± 5% RH

| | | | | | | | | | |
|---|---|---|---|---|---|---|---|---|---|
| Test | Acceptance | | 1^{st} | 3^{rd} | 6^{th} | 9^{th} | 12^{th} | 18^{th} | 24^{th} |
| Parameters | Limit | Initial | Month | Month | Month | Month | Month | Month | Month |

Purity and Related substances by HPLC (% area) by Method A:

| | | | | | | | | | |
|---|---|---|---|---|---|---|---|---|---|
| Purity | NLT 97.0 | 99.3 | 99.0 | 99.0 | 99.0 | 99.1 | 98.7 | 98.6 | 98.3 |
| Impurity at | NMT 0.50 | ND | ND | ND | ND | ND | ND | ND | ND |
| 0.25 RRT | | | | | | | | | |
| Impurity at | NMT 1.30 | 0.14 | 0.2 | 0.26 | 0.21 | 0.35 | 0.44 | 0.66 | 0.95 |
| 0.62 RRT | | | | | | | | | |
| Impurity at | NMT 0.60 | ≤0.05 | ND | 0.06 | ≤0.05 | ND | ≤0.05 | 0.24 | ≤0.05 |
| 0.72 RRT | | | | | | | | | |
| Impurity at | NMT 0.50 | 0.07 | 0.14 | 0.06 | 0.07 | 0.19 | 0.11 | 0.19 | 0.15 |
| 0.86 RRT | | | | | | | | | |
| Any | NMT 0.50 | — | — | 0.07 | — | 0.08 | 0.20 | 0.20 | ≤0.05 |
| single un- | | | | | | | | | (RRT |
| specified | | | | | | | | | 0.37) |
| impurity | | — | — | — | — | — | — | ≤0.05 | 0.24 |
| | | | | | | | | | (RRT |
| | | | | | | | | | 0.42) |
| | | 0.09 | 0.16 | 0.18 | — | — | 0.18 | — | — |
| | | — | — | — | — | — | — | 0.02 | 0.12 |
| | | | | | | | | ≤0.05 | (RRT |
| | | | | | | | | | 0.75) |
| | | 0.16 | 0.21 | 0.15 | 0.20 | 0.20 | 0.10) | — | 0.17 |
| | | | | | | | | | (RRT |
| | | | | | | | | | 0.80) |
| | | 0.07 | 0.09 | 0.07 | 0.09 | — | 0.06 | — | — |
| | | — | — | — | 0.06 | — | 0.07 | ≤0.05 | — |
| | | — | ≤0.05 | ≤ 0.05 | — | — | — | ≤0.05 | ≤0.05 |
| | | | (RRT1. | (RRT1. | | | | | (RRT |
| | | | 21) | 21) | | | | | 1.21) |
| | | — | — | — | — | — | — | ≤0.05 | — |

Chiral analysis by HPLC (% area):

| | | | | | | | | | |
|---|---|---|---|---|---|---|---|---|---|
| Chiral | NLT 98.0 | 99.2 | 99.3 | 99.3 | 99.3 | 99.2 | 99.3 | 99.2 | 99.2 |
| purity | | | | | | | | | |
| RRT about | NMT 1.5 | 0.70 | 0.69 | 0.69 | 0.69 | 0.76 | 0.70 | 0.68 | 0.70 |
| 0.86 (LDL | | | | | | | | | |
| Isomer) | | | | | | | | | |
| Sum of all | NMT 1.0 | 0.08 | 0.06 | 0.05 | 0.05 | 0.05 | ND | 0.09 | 0.09 |
| other | | | | | | | | | |
| isomers | | | | | | | | | |
| (except | | | | | | | | | |
| LDL | | | | | | | | | |
| isomer) | | | | | | | | | |

NMT: not more than;
NLT: not less than;
ND: not detected

Table no: 4 and table no: 5 disclose the purity of the calcium salt of compound of formula (I) and also shows that the calcium salt of compound of formula (I) surprisingly remains stable up to 24 months, both at long-term storage conditions and accelerated storage conditions. The A-1 impurity is constant across 1^{st} month to 24^{th} month at long term storage conditions. Impurities at RRT 0.72 and RRT 0.86 are also observed to be much below the corresponding acceptance limits under both storage conditions. At RRT 0.25, no impurities were detected in either of the storage conditions. In the long-term storage condition, the calcium salt of compound of formula (I) shows purity above 99% and in the accelerated storage condition, calcium salt of compound of formula (I) shows purity in the range of 98.3 to 99.3 across 24 months of storage duration. As measured by chiral HPLC, the LDL isomer impurity (RRT 0.86) was also observed well below the acceptance limit of NMT 1.5% in both storage conditions.

### OTHER EMBODIMENTS

The foregoing disclosure has been described in some detail by way of illustration and example, for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive.

The scope of the invention should, therefore, be determined not with reference to the above description, but should instead be determined with reference to the following appended claims, along with the full scope of equivalents to which such claims are entitled.

1. A calcium salt of compound of formula (I): 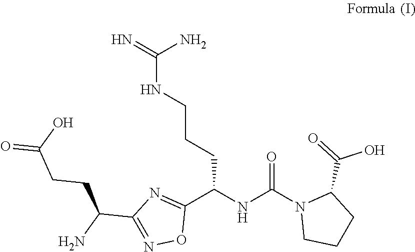
wherein the calcium salt of the compound is substantially free from impurities.

2. The calcium salt of compound of formula (I) according to claim 1, wherein the calcium salt of the compound is substantially free from at least one of the impurities selected from A-1, A-2 and A-3:

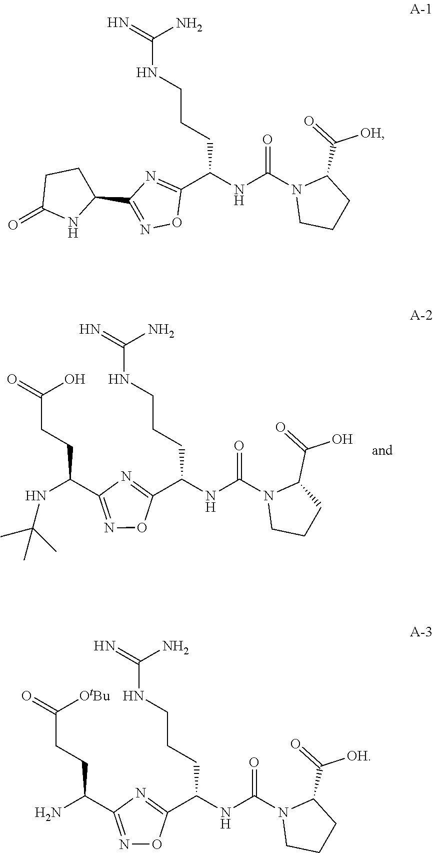 [SMILES: N=C(N)NCCC[C@H](NC(=O)N1CCC[C@H]1C(=O)O)c1nc([C@@H]2CCC(=O)N2)no1 ; CC(C)(C)N[C@@H](CCC(=O)O)c1noc([C@H](CCCNC(=N)N)NC(=O)N2CCC[C@H]2C(=O)O)n1 ; CC(C)(C)OC(=O)CC[C@H](N)c1noc([C@H](CCCNC(=N)N)NC(=O)N2CCC[C@H]2C(=O)O)n1]

3. The calcium salt of compound of formula (I) according to claim 1, wherein the calcium salt of the compound has a purity of at least about 93% as measured by HPLC (% area).

4. (canceled)

5. The calcium salt of compound of formula (I) according to claim 3, wherein the calcium salt of the compound has a purity of at least about 98% to 99.9% as measured by HPLC (% area).

6. The calcium salt of compound of formula (I) according to claim 2, wherein the impurities A-1, A-2 and A-3 are present in a combined amount of not more than about 3%, not more than about 2%, or not more than about 1% as measured by HPLC (% area).

7. The calcium salt of compound of formula (I) according to claim 2, wherein the impurity A-1 is not more than about 1.30% as measured by HPLC (% area).

8. (canceled)

9. The calcium salt of compound of formula (I) according to claim 2, wherein the impurity A-2 is not more than about 0.60% as measured by HPLC (% area).

10. (canceled)

11. The calcium salt of compound of formula (I) according to claim 2, wherein the impurity A-3 is not more than about 0.60% as measured by HPLC (% area).

12. (canceled)

13. The calcium salt of compound of formula (I) according to claim 2, wherein upon storage of the calcium salt of the compound at 2-8 °C the impurity A-1 remains less than about 3% as measured by HPLC (% area) for a period of 12 months.

14. (canceled)

15. The calcium salt of compound of formula (I) according to claim 1, wherein the calcium salt of the compound is substantially free from at least one of the following impurities as measured by HPLC (% area):
a. not more than about 0.50% of impurity at RRT 0.25;
b. not more than about 0.50% of impurity at RRT 0.86;
c. not more than about 0.20% of impurity at RRT 0.56;
d. not more than about 0.30% of impurity at RRT 0.68;
e. not more than about 0.15% of impurity at RRT 1.07;
f. not more than about 0.20% of impurity at RRT 1.21;
g. not more than about 0.20% of impurity at RRT 1.42; or
h. not more than about 0.10% of other single unspecified impurity;
or
a. not more than about 0.50% of impurity at RRT 0.25;
b. not more than about 0.50% of impurity at RRT 0.86; or
c. not more than about 0.50% of any other single unspecified impurity selected from: impurity at RRT 0.56, impurity at RRT 0.68, impurity at RRT 1.07, impurity at RRT 1.21 and impurity at RRT 1.42;
or
a. not more than about 0.08%, preferably not more than about 0.07% of impurity at RRT 0.25;
b. not more than about 0.38%, preferably not more than about 0.24% of impurity at RRT 0.86; or
c. not more than about 0.13%, preferably not more than about 0.10% of any other single unspecified impurity selected from: impurity at RRT 0.56, impurity at RRT 0.68, impurity at RRT 1.07, impurity at RRT 1.21 and impurity at RRT 1.42.

16. The calcium salt of compound of formula (I) according to claim 1, wherein the compound of formula (I) is enantiomerically pure LLL-isomer of the compound of formula (I).

17. The calcium salt of compound of formula (I) according to claim 16, wherein the compound of formula (I) has a chiral purity of at least about 94.6% as measured by chiral HPLC (% area).

18. The calcium salt of compound of formula (I) according to claim 16, wherein the calcium salt of the compound is substantially free from at least one of the following impurities as measured by chiral HPLC (% area):
a. LDL isomer impurity (RRT 0.86) of not more than about 1.90%, preferably not more than about 1.50%, more preferably not more than about 0.78%, even more preferably in the range of about 0.78% to 1.90%;
b. all other isomers not more than about 3.50%, preferably about not more than 1.00%, more preferably not more than about 0.02%, even more preferably in the range of about 0.02% to 3.50%, wherein the isomers are selected from one or more of: DLD isomer impurity, DDL isomer impurity, DLL isomer impurity, DDD isomer impurity, LLD isomer impurity and LDD isomer impurity.

19-30. (canceled)

31. A pharmaceutical composition comprising the calcium salt of compound of formula (I) according to claim 1 and one or more pharmaceutically acceptable excipients.

32. The pharmaceutical composition according to claim 31, wherein upon storage of the composition at 2-8 °C the impurity A-1 remains less than about 3%, preferably less than about 1.3% as measured by HPLC (% area) for a period of 12 months, preferably 18 months, more preferably 24 months, and/or wherein upon storage of the composition at 25 °C/60% RH; the impurity A-1 remains less than about 3%, preferably less than about 1.3% as measured by HPLC (% area) for a period of 12 months, preferably 18 months, more preferably 24 months.

33. (canceled)

34. The pharmaceutical composition according to claim 31, wherein the composition is a solid oral dosage form, oral liquid dosage form, powder for oral suspension.

35-36. (canceled)

37. A method of treating cancer comprising administering a therapeutically effective amount of the calcium salt of compound of formula (I) according to claim 1 to a patient in need thereof, wherein the cancer is selected from
breast cancer, lung cancer colorectal cancer, prostate cancer, ovarian cancer, oropharyngeal cancer, nasopharyngeal cancer, cervical cancer, neuroendocrine cancer, gastrointestinal cancer, stomach cancer, pancreatic cancer, liver cancer, or
a hematological cancer selected from Acute Myeloid Leukemia (AML) or Myelodysplastic syndrome (MDS), or
a cancer associated fibrosis selected from biliary cancer, cholangiocarcinoma, bladder cancer, colorectal cancer, colon cancer, rectal cancer, colorectal adenocarcinoma, brain cancer, meningioma, glioma, astrocytoma, oligodendroglioma, medulloblastoma, breast cancer, adenocarcinoma of the breast, inflammatory carcinoma of the breast, papillary carcinoma of the breast, medullary carcinoma of the breast, kidney cancer nephroblastoma/Wilms' tumor, renal cell carcinoma, cervical cancer, squamous cell carcinoma of the cervix, cervical adenocarcinoma, gastric cancer, esophageal cancer, gastrointestinal stromal tumor (GIST), head and neck cancer, head and neck squamous cell carcinoma, oral cancer, oral squamous cell carcinoma (OSCC), keloids, leukemia, acute lymphocytic leukemia (ALL), acute myelogenous leukemia (AML), chronic myelogenous leukemia (CML), chronic lymphocytic leukemia (CLL), lung cancer bronchogenic carcinoma, small cell lung cancer (SCLC), non-small cell lung cancer (NSCLC), adenocarcinoma of the lung, liver cancer, hepatocellular cancer (HCC), malignant hepatoma, lymphoma, Hodgkin lymphoma (HL), non-Hodgkin lymphoma (NHL), follicular lymphoma, diffuse large B-cell lymphoma (DLBCL), mantle cell lymphoma (MCL), medulloblastoma, multiple myeloma (MM), myelodysplasia syndrome (MDS), myeloproliferative disorder (MPD), polycythemia vera (PV), essential thrombocythemia (ET), agnogenic myeloid metaplasia (AMM), primary myelofibrosis (PMF), chronic neutrophilic leukemia (CNL), hypereosinophilic syndrome (HES), neuroblastoma, neurofibroma, neurofibromatosis (NF) type 1, neurofibromatosis (NF) type 2, schwannomatosis, skin cancer, squamous cell carcinoma (SCC), keratoacanthoma (KA), melanoma, basal cell carcinoma (BCC), dermatofibroma, osteosarcoma, ovarian cancer, cystadenocarcinoma, ovarian embryonal carcinoma, ovarian adenocarcinoma, neuroendocrine cancer, gastroenteropancreatic neuroendocrine tumor (GEP-NET), carcinoid tumor, pancreatic cancer, pancreatic adenocarcinoma, intraductal papillary mucinous neoplasm (IPMN), prostate cancer, prostate adenocarcinoma, soft tissue tumors, angiolipoma, angioleiomyoma, malignant fibrous histiocytoma (MFH), liposarcoma, malignant peripheral nerve sheath tumor (MPNST), chondrosarcoma, fibrosarcoma, myosarcoma, osteosarcoma, and tumors associated with desmoplasia.

38-43. (canceled)

44. The method according to claim 37, wherein the calcium salt of compound of formula (I) is administered at a total daily dose not exceeding 800 mg.

45. The method according to claim 37, wherein the calcium salt of compound of formula (I) is administered to the patient in need once or twice daily.

46. A compound of formula A-2:

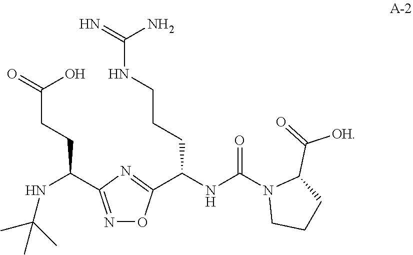

47. A compound of formula A-3: [chemical structure]
